(19) United States
(12) Patent Application Publication
(10) Pub. No.: US 2006/0193797 A1
(43) Pub. Date: Aug. 31, 2006

(54) Chroman derivatives as lipoxygenase inhibitors

(75) Inventors: Wei Zhang, Santa Clara, CA (US); Jian Chen, San Jose, CA (US); Sekhar Boddupalli, San Jose, CA (US)

Correspondence Address: FOLEY & LARDNER LLP, 1530 PAGE MILL ROAD, PALO ALTO, CA, 94304, US

(73) Assignee: Galileo Pharmaceuticals, Inc

(21) Appl. No.: 11/349,813

(22) Filed: Feb. 7, 2006

Related U.S. Application Data
(60) Provisional application No. 60/656,644, filed on Feb. 25, 2005.

(51) Int. Cl. A61K 8/49; C07D 311/04; A61K 31/353

(52) U.S. Cl. 424/59; 514/456; 549/404; 549/403; 549/23; 546/153

(57) ABSTRACT

The present invention is concerned with certain novel derivatives of Formula I: 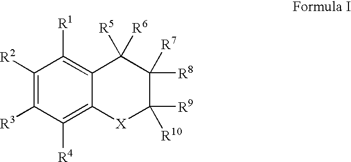
wherein X and R¹ to R¹⁰ are as described in the specification, and where either R⁵ is OH, —NR^{d}OR^{a} or —NR^{d}—NR^{b}R^{c}, or R⁷ is —NR^{d}OR^{a} or —NR^{d}—NR^{b}R^{c}, or C═R⁷R⁸ is C═NOR^{a} or C═N—NR^{b}R^{c}, which may be useful in the manufacture of pharmaceutical compositions for treating disorders mediated by lipoxygenases. They may also be useful in the manufacture of skin care and/or pharmaceutical compositions for the treatment of lipoxygenase mediated disorders.

### CROSS REFERENCE TO RELATED APPLICATIONS

This application claims the benefit under 35 U.S.C. §119(e) of U.S. Provisional Application Ser. No. 60/656,644 filed on Feb. 25, 2005, which is hereby incorporated by reference in its entirety.

### BACKGROUND INFORMATION

The present invention relates to certain novel chroman, thiochroman, and indoline derivatives of Formula I as depicted below, skin care and/or pharmaceutical compositions containing them, and their uses as therapeutic agents, and syntheses therefore. Their uses as therapeutic agents that may act as lipoxygenase inhibitors include, but are not limited to, prevention or treatment of diseases involving apoptosis in cancer cells; diseases involving hypoxia or anoxia; diseases involving inflammation; disorders of the airways; diseases involving neurodegeneration and neuroinflammation; and diseases involving the autoimmune system. Their uses as skin care agents include, but are not limited to, prevention or treatment of inflammatory disorders of the skin such as acne, dermatitis and psoriasis, psoriasis, eczema, skin irritation, and the like.

The use of compounds having a chroman moiety as lipoxygenase inhibitors has been disclosed, for example, in U.S. Pat. No. 5,059,609; U.S. Pat. No. 4,950,684; U.S. Pat. No. 5,015,661; U.S. Pat. No. 4,780,469; U.S. Pat. No. 5,591,772; U.S. Pat. No. 5,925,673; U.S. Pat. No. 5,250,547; U.S. Pat. No. 5,393,775; U.S. Pat. No. 4,814,346; U.S. Pat. No. 5,939,452, U.S. Pat. No. 6,051,601; U.S. Pat. No. 6,117,874; and U.S. Pat. No. 6,133,286.

Arachidonic acid is an essential fatty acid that exists within the cell membrane and can be released from phospholipids by the action of phospholipase. The released arachidonic acid is metabolized through three major enzymatic pathways, i.e. the lipoxygenase pathway, to form substances such as prostaglandins which are associated with inflammatory responses, and thromboxanes which are associated with the formation of thrombus, or leukotrienes which induce allergic reactions.

Lipoxygenases are non-heme iron-containing enzymes that catalyze the oxidation of polyunsaturated fatty acids and esters thereof. They were originally classified based on their substrate specificity for insertion of molecular oxygen into arachidonic acid at carbon positions 5, 12 and 15, but more recently a phylogenetic classification is being used. This separates the mammalian enzymes in four main subtypes, 5-Lipoxygenase, 12/15-Lipoxygenases, platelet 12-Lipoxygenases and epidermis-type lipoxygenases. The 12/15 family of lipoxygenases includes two sub-families with a high degree of sequence homology, the reticulocyte 15-Lipoxygenases (found in rabbit and humans) and the leukocyte 12-Lipoxygenases (found in mouse, pig, rat, and rabbit). This type of lipoxygenase shares more homology to reticulocyte 15-Lipoxygenase and leukocyte 12-Lipoxygenase, than to platelet 12-Lipoxygenases.

It is believed that oxidative metabolites of the 12/15-Lipoxygenase or the 15-Lipoxygenase cascade have been implicated in the potentiation of thrombin induced platelet activation (Setty et al. Blood, (1992), 2765-2773); in the progression of various cancers (Kelavkar et al, Curr. Urol. Rep. Vol. 3 no. 3 (2002): pp. 207-214) and related pathologies (Tisdale et al., Science Vol. 289 no. 5488 (2000) pp. 2293-4). It has also been shown that treatment with a 15-Lipoxygenase inhibitor suppresses atherogenesis in rabbits fed a high-fat diet (Bocan et al., Atherosclerosis, Vol. 136 (1998) pp. 203-16). There is increasing evidence that certain lipoxygenase enzymes are involved in the pathogenesis and acceleration of atherosclerosis by inducing oxidation of LDL to its atherogenic form (Sparrow, C. P., et al., J. Lipid Res. Vol. 29 (1988) pp. 745-753. and Steinberg, D., New Eng. J. Med. Vol. 320(1989) pp. 915-924). It has also been reported that 12-Lipoxygenase enzyme plays a role in mediating angiotensin II induced vascular and adrenal actions (Natarajan, R., et al., Endocrinology Vol. 131 (1992) pp. 1174-1180). Recent studies (Klein, R. et al., Science Vol. 303 no. 5655 (2004) 329-332) have also shown the role of 15-Lipoxygenase enzyme in the regulation of bone density.

The enzyme 5-Lipoxygenase converts arachidonic acid to 5-hydroperoxyeicosatetraenoic acid (5-HPETE). This is the first step in the metabolic pathway yielding 5-hydroxyeicosatetraenoic acid (5-HETE) and the important class of mediators, the leukotrienes. Evidence of the role of leukotrienes in the pathology of certain diseases has been described, for example in Cloud et al., J. Allergy Clin. Immunol., Vol. 79 (1987) pp. 256 (asthma); Turnbull et al., Lancet II, (1977) pp. 526-9 (chronic bronchitis); Cromwell et al., Lancet II, (1981) pp. 164-5 (cystic fibrosis); Davidson et al., J. Pharm. Pharmacol. Vol. 34 no. 61(982) pp. 410 (rheumatoid arthritis); Rae et al., Lancet. Vol. 2 no. 8308 (1982) pp. 1122-4. Cook et al., J. Pharmacol. Exp. Ther., 235, (1985) pp. 470-474 (cardiovascular conditions); Tsuji et al., Biochem. Pharmacol. Vol. 55 no. 3: (1998); pp. 297-304 (dermatitis such as psoriasis).

It has also been shown in co-owned U.S. application Ser. No. 11/251,423 filed Oct. 13, 2005, titled Methods for Treating Diabetes, herein incorporated by reference in its entirety, that dual 5-Lipoxygenase and 12/15-Lipoxygenase inhibitors or 5-Lipoxygenase and 15-Lipoxygenase inhibitors are superior in the prevention of treatment of subjects susceptible to diabetes, are able to improve glucose control in animal models of diabetes, and have demonstrated a significant lowering of the baseline serum glucose levels compared to selective 5-Lipoxygenase, 15-Lipoxygenase and 12/15-Lipoxygenase inhibitors.

The compositions, formulations and methods of this invention are particularly applicable in preventing and/or treating diseases or disorders mediated, at least in part, by one or more lipoxygenase enzymes, such as 5-Lipoxygenase enzyme and/or 12/15-Lipoxygenase enzyme.

### SUMMARY OF THE INVENTION

The present invention is concerned with certain novel derivatives of Formula I, which may be useful in the manufacture of pharmaceutical compositions for treating disorders mediated by lipoxygenases and inflammatory skin conditions.

In a first aspect, the present invention concerns the compounds represented by Formula I: 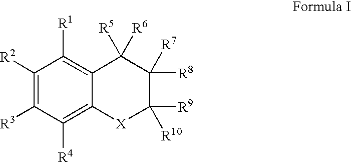
wherein,
X is O, S(O)₀₋₂, or NR;
R¹ and R⁴ are independently selected from the group consisting of hydrogen, alkyl, alkenyl, alkynyl, cycloalkyl, halogen, nitro, cyano, amino, aminosulfonyl, sulfanyl, aryl, heterocyclyl, hydroxy, alkoxy, carboxy, alkoxycarbonyl, and amido; with the proviso that no more than one of R¹ and R⁴ is hydrogen;
R² is selected from the group consisting of hydroxy, alkoxy, —O-alkenyl, —O-acyl, —O-alkylene-amino, —O—C(O)-alkylene-COOR^{b}, —O—C(O)-alkylene-amino, —O—C(O)-alkylene-heterocyclyl, —O-glucoside, —O-phosphoryl, —O-alkylene-phosphoryl, or —O—C(O)-AA, wherein AA is amino acid, or a di-, tri-, or tetra-peptide;
R³ is selected from the group consisting of alkyl, alkenyl, alkynyl, cycloalkyl, halogen, nitro, cyano, amino, aminosulfonyl, sulfanyl, aryl, heterocyclyl, alkoxy, carboxy, alkoxycarbonyl, and amido; or
R³ and R⁴ together with the atoms to which they are attached form a cycloalkyl ring, aryl ring or a heterocyclic ring;
R⁵ and R⁶ are independently selected from the group consisting of hydrogen, alkyl, cycloalkyl, hydroxy, —NR^{d}OR^{a}, and —NR^{d}—NR^{b}R^{c};
R⁷ and R⁸ are
   independently selected from the group consisting of hydrogen, alkyl, cycloalkyl, —NR^{d}OR^{a}, and —NR^{d}—NR^{b}R^{c}; or
   together with the carbon atom to which they are attached form a C═NOR^{a} or a C═N—NR^{b}R^{c} group;
R⁹ is selected from the group consisting of hydrogen, alkyl and cycloalkyl;
R¹⁰ is alkyl or cycloalkyl;
R is selected from the group consisting of hydrogen, alkyl, cycloalkyl, alkenyl, alkynyl, acyl, aminocarbonyl, heterocyclyl, and aryl;
R^{a} is selected from the group consisting of alkyl, cycloalkyl, alkenyl, acyl, heterocyclyl, and aryl; and
R^{b} and R^{c} are
   independently selected from the group consisting of hydrogen, alkyl, cycloalkyl, alkenyl, acyl, aminocarbonyl, heterocyclyl and aryl; or
   together with the nitrogen atom to which they are attached form an optionally substituted, saturated or unsaturated 3-8 membered ring optionally incorporating 1 to 3 N, O or S atoms; and
R^{d} is hydrogen or alkyl;
   with the proviso that one of the following is present
      R⁵ is OH, —NR^{d}OR^{a} or —NR^{d}—NR^{b}R^{c}; or
      R⁷ is —NR^{d}OR^{a} or —NR^{d}—NR^{b}R^{c}; or
      R⁷ and R⁸ together with the carbon atom to which they are attached form a C═NOR^{a} or a C═N—NR^{b}R^{c} group;
         or single stereoisomers, mixtures of stereoisomers, or pharmaceutically acceptable salts thereof.

In one embodiment, R² is hydroxy, and in another embodiment R² is hydroxy and R¹, R³, and R⁴ are independently of each other hydrogen, halogen, or alkyl. In yet another embodiment CR⁷R⁸ is C═NOR^{a}; and in another embodiment CR⁷R⁸ is C═N—NR^{b}R^{c}. In another embodiment R⁵ is —NR^{d}OR^{a}; in another embodiment R⁵ is —NR^{d}—NR^{b}R^{c}; and in yet another embodiment R⁵ is OH and R⁶ is hydrogen. In another embodiment R⁷ is —NR^{d}OR^{a}; and in another embodiment R⁷ is —NR^{d}—NR^{b}R^{c}. In some embodiments X is O; in other embodiments X is S; and in other embodiments X is NR, wherein R is aryl, heterocyclyl, or alkyl substituted with amido, sulfonylamino, aminosulfonyl or aryl, and in another embodiment R is —(CH₂)₂₋₆—NR^{d}S(O)₂-aryl, —(CH₂)₂₋₆—S(O)₂NR^{d}-aryl; —(CH₂)₂₋₆NR^{d}C(O)-aryl or —(CH₂)₂₋₆—C(O)NR^{d}-aryl; illustrated by alkylbenzenesulfonaminoethyl, or alkylbenzenesulfonaminopropyl.

In other embodiments, when R² is hydroxy and X is O then R¹, R³, and R⁴ are independently selected from the group consisting of hydrogen, halogen, and alkyl. In other embodiments when R² is hydroxy and X is S, then R¹, R³, and R⁴ are independently selected from the group consisting of hydrogen, halogen, and alkyl. In yet other embodiments, when R² is hydroxy and X is NR, then R¹, R³, and R⁴ are selected from the group consisting of hydrogen, halogen, or alkyl.

In another aspect, the invention relates to a pharmaceutical composition containing a therapeutically effective amount of a compound of Formula I. In some examples, the pharmaceutical compositions comprise a compound of Formula I and a pharmaceutically acceptable excipient and the compound is selected from the illustrative compounds and stereoisomers, mixture of stereoisomers or pharmaceutically acceptable salts thereof.

In another aspect, the invention relates to a method of inhibiting one or more lipoxygenase enzymes selected from 5-lipoxygenase, 15-lipoxygenase, 12/15-lipoxygenase enzymes, and combinations thereof with the compounds of the invention. In some embodiments, the compound inhibits the 5-lipoxygenase enzyme, and in other embodiments the compound inhibits both 5- and 15-lipoxygenase enzymes or both 5- and 12/15-lipoxygenase enzymes.

In some embodiments, the invention relates to a method of treating a subject with a lipoxygenase mediated disorder such as, but not limited to, apoptosis in cancer cells including prostatic cancer, gastric cancer, breast cancer, pancreatic cancer, colorectal or esophageal cancer and airways carcinoma; diseases involving hypoxia or anoxia including atherosclerosis, myocardial infarction, cardiovascular disease, heart failure (including chronic and congestive heart failure), cerebral ischemia, retinal ischemia, myocardial ischemia, post surgical cognitive dysfunction and other ischemias; diseases involving inflammation, including diabetes, arterial inflammation, inflammatory bowel disease, Crohn's disease, renal disease, pre-menstrual syndrome, asthma, allergic rhinitis, gout, cardiopulmonary inflammation, rheumatoid arthritis, osteoarthritis, muscle fatigue, disorders of the airways including asthma, chronic bronchitis, human airway carcinomas, mucus hypersecretion, chronic obstructive pulmonary disease (COPD) pulmonary fibrosis caused by chemotherapy or other drugs, idiopathic pulmonary fibrosis, cystic fibrosis and adult respiratory distress syndrome; diseases involving central nervous system (CNS) disorders including psychiatric disorders including anxiety and depression; neurodegeneration and neuroinflammation including Alzheimer's, dementia and Parkinson's disease; peripheral neuropathy including spinal chord injury, head injury and surgical trauma, and allograft tissue and organ transplant rejection; diseases involving the autoimmune system including rheumatoid arthritis, and diabetes; and disorders involving bone loss or bone formation. In an illustrative example, the invention relates to a method of treating a subject with a lipoxygenase mediated disorder, such as but not limited to diabetes, arthritis, rheumatoid arthritis, chronic obstructive pulmonary disease (COPD), asthma, allergic rhinitis, Crohn's disease, and/or atherosclerosis.

In another aspect, the lipoxygenase disorder is selected from inflammatory disorders of the skin including dermatitis, including atopic, contact, and allergic dermatitis, xerosis, eczema, rosacea, seborrhea, psoriasis, atherosclerosis, thermal and radiation burns, acne, oily skin, wrinkles, excessive cellulite, excessive pore size, intrinsic skin aging, photo aging, photo damage, harmful UV damage, keratinization abnormalities, irritation including retinoid induced irritation, hirsutism, alopecia, dyspigmentation, inflammation due to wounds, scarring or stretch marks, loss of elasticity, and skin atrophy.

Another aspect of the invention, concerns a pharmaceutical composition comprising at least one compound of Formula IA: 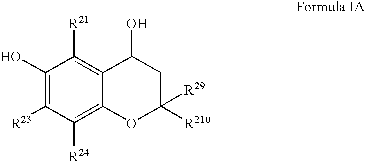
wherein,
R²¹, R²⁴ and R²⁹ are independently selected from the group consisting of hydrogen, alkyl and cycloalkyl;
with the proviso that no more than one of R²¹ and R²⁴ is hydrogen; and
R²³ and R²¹⁰ are independently alkyl or cycloalkyl;
or single stereoisomers, mixtures of stereoisomers, or pharmaceutically acceptable salts thereof; and a pharmaceutically acceptable excipient. In some embodiments the pharmaceutical composition comprises compounds wherein R²¹ and R²³ are C₁₋₄ alkyl, R²⁴ is hydrogen, and R²⁹ and R²¹⁰ are both methyl. In some embodiments, the pharmaceutical compositions comprise at least one compound selected from 5,7-diethyl-2,2-dimethylchroman-4,6-diol; 5-ethyl-7-isopropyl-2,2-dimethylchroman-4,6-diol; 7-isopropyl-2,2,5-trimethylchroman-4,6-diol; 2,2,7,8-tetramethylchroman-4,6-diol; and 2,2,5,7,8-pentamethylchroman-4,6-diol
or stereoisomers, mixture of stereoisomers or pharmaceutically acceptable salts thereof; and a pharmaceutically acceptable excipient.

In another aspect, the invention relates to a skin care composition comprising at least one compound of Formula IA. In one embodiment, R²³ and R²⁴ are independently C₁₋₄ alkyl, R²¹ is hydrogen or methyl, and R²⁹ and R²¹⁰ are both methyl.

In another aspect, the invention relates to a skin care composition comprising as the active component 2,2,5,7,8-pentamethylchroman-4,6-diol, 2,2,7,8-tetramethylchroman-4,6-diol, or mixtures thereof, admixed with a cosmetically acceptable carrier.

In the skin care composition aspects of the invention, the composition can further comprise at least one agent selected from the group consisting of:
(i) a skin protectant active ingredient selected from the group consisting of allantoin, aluminum hydroxide gel, calamine, cocoa butter, cod liver oil, colloidal oatmeal, dimethicone, glycerin, hard fat, kaolin, lanolin, mineral oil, petrolatum, soy products, sodium bicarbonate, topical starch, white petrolatum, zinc acetate, and/or zinc oxide;
(ii) an external analgesic, anesthetic or antipruritic ingredient selected from the group consisting of benzocaine, butamaben picrate, dibucaine, dibucaine hydrochloride, dimethiosoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium resorcinol, tripelennamine hydrochloride, aspirin, hydrocortisone, hydrocortisone acetate, and/or diphenydramine hydrochloride;
(iii) a keratolytic agent selected from the group consisting of salicylic acid or esters thereof, benzoyl peroxide, resorcinol, colloidal sulfur, selenium disulphide, sulfur and combinations thereof; and
(iv) a retinoid selected from the group consisting of retinol, retinoic acid and esters thereof.

In some embodiments, the agent is a retinoid or is a soy product.

In other embodiments, the composition further comprises at least one other agent selected from the group consisting of other sebum suppressant agents, antimicrobial agents, antibacterial agents, antifungal agents, antioxidants, buffering agents, sunscreens, cosmetic agents, fragrances, lubricants, moisturizers, drying agents, and thickening agents. In other embodiments, the compositions of the present invention may additionally contain at least one agent selected from the group consisting of a bacterial lipase inhibitor, a bacterial proliferation inhibitor, an anti-inflammatory agent and a keratolytic agent.

The invention also relates to a method of treating a lipoxygenase mediated condition comprising administering a cosmetically effective amount of the skin care compositions described above, wherein the condition is selected from the group consisting of dermatitis, including atopic, contact, and allergic dermatitis, xerosis, eczema, rosacea, seborrhea, psoriasis, atherosclerosis, thermal and radiation burns, acne, oily skin, wrinkles, excessive cellulite, excessive pore size, intrinsic skin aging, photo aging, photo damage, harmful UV damage, keratinization abnormalities, irritation including retinoid induced irritation, hirsutism, alopecia, dyspigmentation, inflammation due to wounds, scarring or stretch marks, loss of elasticity, and skin atrophy. In some embodiments, the condition is irritation such as retinoid induced irritation or acne.

Another aspect of the invention, concerns a pharmaceutical composition comprising at least one compound of Formula IB: 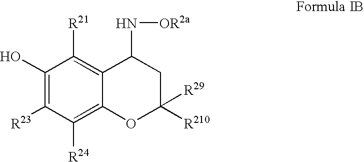
wherein,
R²¹, R²⁴ and R²⁹ are independently selected from the group consisting of hydrogen, alkyl or cycloalkyl;
with the proviso that no more than one of R²¹ and R²⁴ is hydrogen;
R²³ and R²¹⁰ are independently alkyl or cycloalkyl; and
R^{2a} is alkyl or cycloalkyl;
or single stereoisomers, mixtures of stereoisomers, or pharmaceutically acceptable salts thereof, admixed with a pharmaceutically acceptable excipient.

In some embodiments the pharmaceutical compositions comprise at least one compound selected from 4-methoxyamino-2,2,5,7,8-pentamethyl-chroman-6-ol; 4-(methoxyamino)-2,2,7,8-tetramethylchroman-6-ol; 5,7-diethyl-4-(methoxyamino)-2,2,8-trimethylchroman-6-ol; 7-isopropyl-4-(methoxyamino)-2,2,5-trimethylchroman-6-ol; and 7-isopropyl-4-(methoxyamino)-2,2,5-trimethylchroman-6-ol; or stereoisomers, mixture of stereoisomers or pharmaceutically acceptable salts thereof, and a pharmaceutically acceptable excipient.

The invention also relates to methods of treating a subject with a lipoxygenase mediated condition comprising administering to said subject a therapeutically effective amount of a pharmaceutical composition comprising one or more compounds of Formula IA or IB admixed with a pharmaceutically acceptable excipient. In some aspects, the conditions are selected from the group consisting of diabetes, arthritis, rheumatoid arthritis, chronic obstructive pulmonary disease (COPD), asthma, allergic rhinitis, or atherosclerosis. In other aspects, the conditions are selected from the group consisting of dermatitis, eczema, skin irritation or psoriasis.

In another aspect, the invention relates to novel compounds represented by Formula IA or Formula IB. In some embodiments, the compounds are represented by Formula IA or Formula IB wherein R²¹ and R²³ are C₁₋₄ alkyl, R²⁴ is hydrogen, and R²⁹ and R²¹⁰ are methyl. In other embodiments, of Formula IA, R²¹ is hydrogen or methyl, and R²³ and R²⁴ are C₁₋₄ alkyl, and R²⁹ and R²¹⁰ are methyl.

Another aspect of the invention concerns a compound selected from
6-hydroxy-2,2,5,7,8-pentamethyl-chroman-4-one O-methyl-oxime;
6-hydroxy-2,2,5,7,8-pentamethyl-thiochroman-4-one O-methyl-oxime;
4-methoxyamino-2,2,5,7,8-pentamethyl-chroman-6-ol;
6-hydroxy-2,2,5,7,8-pentamethyl-2,3-dihydro-4H-chromen-4-one dimethylhydrazone;
6-hydroxy-2,2,5,7,8-pentamethylchroman-3-one O-methyl oxime;
8-fluoro-4-(methoxyamino)-2,2,5,7-tetramethylchroman-6-ol;
4-(methoxyamino)-2,2,7,8-tetramethylchroman-6-ol;
4-(ethoxyamino)-2,2,7,8-tetramethylchroman-6-ol;
5,7-diethyl-4-(methoxyamino)-2,2,8-trimethylchroman-6-ol;
7-isopropyl-4-(methoxyamino)-2,2,5-trimethylchroman-6-ol;
5-ethyl-7-isopropyl-4-(methoxyamino)-2,2-dimethylchroman-6-ol
4-(methoxyamino)-2,2,5,7,8-pentamethyl-1,2,3,4-tetrahydroquinolin-6-ol;
1-(4-hydroxyphenyl)-4-(methoxyamino)-2,2,5,7,8-pentamethyl-1,2,3,4-tetrahydroquinolin-6-ol;
4-(2,2-dimethylhydrazinyl)-2,2,5,7,8-pentamethyl-1,2,3,4-tetrahydroquinolin-6-ol;
4-(2,2-dimethylhydrazinyl)-1-(4-hydroxyphenyl)-2,2,5,7,8-pentamethyl-1,2,3,4-tetrahydroquinolin-6-ol
2,2,5,7,8-pentamethylchroman-4,6-diol
2,2,7,8-tetramethylchroman-4,6-diol;
5,7-diethyl-2,2-dimethylchroman-4,6-diol;
5-ethyl-7-isopropyl-2,2-dimethylchroman-4,6-diol; and
7-isopropyl-2,2,5-trimethylchroman-4,6-diol;
   and single stereoisomers, mixtures of stereoisomers, or pharmaceutically acceptable salts thereof.

In some embodiments the compound is selected from 4-methoxyamino-2,2,5,7,8-pentamethyl-chroman-6-ol; 4-(methoxyamino)-2,2,7,8-tetramethylchroman-6-ol; 5,7-diethyl-4-(methoxyamino)-2,2,8-trimethylchroman-6-ol; 7-isopropyl-4-(methoxyamino)-2,2,5-trimethylchroman-6-ol; and 7-isopropyl-4-(methoxyamino)-2,2,5-trimethylchroman-6-ol and single stereoisomers, mixtures of stereoisomers, or pharmaceutically acceptable salts thereof. In other embodiments the compound is selected from 2,2,5,7,8-pentamethylchroman-4,6-diol; 2,2,7,8-tetramethylchroman-4,6-diol; 5,7-diethyl-2,2-dimethylchroman-4,6-diol; 5-ethyl-7-isopropyl-2,2-dimethylchroman-4,6-diol; and 7-isopropyl-2,2,5-trimethylchroman-4,6-diol; or stereoisomers, mixture of stereoisomers or pharmaceutically acceptable salts thereof.

Another aspect of this invention is the processes for preparing compounds of Formula I and is set forth in “Detailed Description of the Invention.”

### DETAILED DESCRIPTION OF THE INVENTION

Definitions

As used in the present specification, the following words and phrases are generally intended to have the meanings as set forth below, except to the extent that the context in which they are used indicates otherwise.

The term “optional” or “optionally” means that the subsequently described event or circumstance may or may not occur, and that the description includes instances where said event or circumstance occurs and instances in which it does not.

It will be understood by those skilled in the art with respect to any group containing one or more substituents that such groups are not intended to introduce any substitution or substitution patterns that are sterically impractical and/or physically non-feasible.

The term “acyl” refers to the groups —C(O)—H, —C(O)-(alkyl), —C(O)-(cycloalkyl), —C(O)-(alkenyl), —C(O)-(cycloalkenyl), —C(O)-(aryl), and —C(O)-(heterocyclyl).

The term “acyloxy” refers to the moiety —O-acyl, including, for example, —O—C(O)-alkyl.

The term “alkenyl” refers to a monoradical branched or unbranched, unsaturated or polyunsaturated hydrocarbon chain, having from about 2 to 20 carbon atoms, for example 2 to 10 carbon atoms. This term is exemplified by groups such as ethenyl, but-2-enyl, 3-methyl-but-2-enyl (also referred to as “prenyl”), octa-2,6-dienyl, 3,7-dimethyl-octa-2,6-dienyl (also referred to as “geranyl”), and the like. The term also includes substituted alkenyl groups, and refers to an alkenyl group in which 1 or more, for example, 1 to 3, hydrogen atoms is replaced by a substituent independently selected from the group: ═O, ═S, acyl, acyloxy, alkoxy, amino (wherein the amino group may be a cyclic amine), aryl, heterocyclyl, carboxyl, carbonyl, amido, cyano, cycloalkyl, cycloalkenyl, halogen, hydroxyl, nitro, sulfamoyl (—SO₂NH₂), sulfanyl, sulfinyl (—S(O)H), sulfonyl (—SO₂H), and sulfonic acid (—SO₂OH). One of the optional substituents for alkenyl may be heterocyclyl, exemplified by 2-quinolyl-2-vinyl.

The term “alkenylene” refers to a diradical derived from the above defined monoradical, alkenyl.

The term “alkoxy” refers to the groups: —O-alkyl, —O-alkenyl, —O-cycloalkyl, —O-cycloalkenyl, and —O-alkynyl. Alkoxy groups that are —O-alkyl include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like. The term “alkoxy” also includes substituted alkoxy groups and refers to the groups —O-(substituted alkyl), —O-(substituted alkenyl), —O-(substituted cycloalkyl), —O-(substituted cycloalkenyl), —O-(substituted alkynyl) and —O-(optionally substituted alkylene)-alkoxy.

The term “alkyl” refers to a monoradical branched or unbranched saturated hydrocarbon chain having from about 1 to 20 carbon atoms. The term “alkyl” also means a combination of linear or branched and cyclic saturated hydrocarbon radical consisting solely of carbon and hydrogen atoms. This term is exemplified by groups such as methyl, ethyl, n-propyl, iso-propyl, n-butyl, iso-butyl, n-hexyl, n-decyl, tetradecyl, and the like. The term “alkyl” also includes substituted alkyl and refers to an alkyl group in which 1 or more, such as 1 to 5, hydrogen atoms is replaced by a substituent independently selected from the group: ═O, ═S, acyl, acyloxy, alkoxy, alkoxyamino, hydroxyamino, amino (wherein the amino group may be a cyclic amine), aryl, heterocyclyl, azido, carboxyl, alkoxycarbonyl, amido, cyano, cycloalkyl, cycloalkenyl, halogen, hydroxyl, nitro, sulfonylamino, aminosulfonyl, sulfanyl, sulfinyl, sulfonyl, and sulfonic acid. One of the optional substituents for alkyl may be hydroxy or amino, exemplified by hydroxyalkyl groups, such as 2-hydroxyethyl, 3-hydroxypropyl, 3-hydroxybutyl, 4-hydroxybutyl, and the like; dihydroxyalkyl groups (glycols), such as 2,3-dihydroxypropyl, 3,4-dihydroxybutyl, 2,4-dihydroxybutyl, and those compounds known as polyethylene glycols, polypropylene glycols and polybutylene glycols, and the like; or aminoalkyl groups exemplified by groups such as aminomethyl, dimethylaminomethyl, diethylaminomethyl, ethylaminomethyl, piperidinylmethyl, morpholinylmethyl, and the like. Another substituent for alkyl may be halogen, such as trifluoromethyl. Another substituent may be hydroxyamino or alkoxyamino, exemplified by groups such as hydroxyaminomethyl, methoxyaminomethyl or ethoxyaminomethyl. Another substituent may be sulfanyl, exemplified by groups such as methyl(2-methylthioacetate). Another substituent may be aryl or heterocyclyl exemplified by methylbenzoate, propylisoindoline-1,3-dione, quinoline-methyl or 2-quinolyl-2-ethyl. Another substituent may be amido, aminosulfonyl or sulfonylamino, exemplified by 4-propylbenzensulfonamide-2-ethyl; 4-methylbenzene-sulfonamide-2-ethyl, 4-propylbenzensulfonamide-3-propyl; 4-methylbenzenesulfonamide-3-propyl, or methyl-N-methylacetamide. Another substituent may be aminocarbonyloxy (—OC(O)amino), such as —OC(O)NH₂ or —OC(O)-substituted amino.

The term “alkylene” refers to a diradical alkyl group, whereby alkyl is as defined above.

The term “alkynyl” refers to a monoradical branched or unbranched, unsaturated or polyunsaturated hydrocarbon chain, having from about 2 to 20 carbon atoms, for example 2 to 10 carbon atoms and comprising at least one triple bond, and preferably 1 to 3. The term also includes substituted alkynyl groups, and refers to an alkynyl group in which 1 or more hydrogen atoms is replaced by a substituent independently selected from the group: acyl, acyloxy, alkoxy, amino (wherein the amino group may be a cyclic amine), aryl, heterocyclyl, carboxyl, carbonyl, amido, cyano, cycloalkyl, cycloalkenyl, halogen, hydroxyl, nitro, sulfamoyl, sulfanyl, sulfinyl, sulfonyl, and sulfonic acid.

The term “amido” refers to the moieties —C(O)—NR¹⁰⁰R¹⁰¹ and —NR¹⁰⁰C(O)R¹⁰¹, wherein R¹⁰⁰ and R¹⁰¹ are independently selected from the group consisting of hydrogen, alkyl, substituted alkyl, alkenyl, alkynyl, cycloalkyl, cycloalkenyl, cycloalkynyl, and heterocyclyl, provided that R¹⁰⁰ and R¹⁰¹ are not aryl or heteroaryl.

The term “amino” refers to the group —NH₂ as well as to the substituted amines such as —NHR^{x} or —NR^{x}R^{x} where each R^{x} is independently selected from the group: alkyl, cycloalkyl, alkenyl, cycloalkenyl, alkynyl, aryl, heterocyclyl, acyl, optionally substituted alkoxy, carboxy and alkoxycarbonyl, and where —NR^{x}R^{x} may also be a cyclic saturated or unsaturated amine, optionally incorporating one or more, for example 1 to 3, additional atoms chosen form N, O or S, and optionally substituted with a substituent selected from the group consisting of ═O, ═S, alkyl, hydroxy, acyloxy, halo, cyano, nitro, sulfanyl, alkoxy, and phenyl. This term is exemplified by such groups as amino, cyclopropylamino, dimethylamino, diethylamino, hexylamino. The term “cyclic amine” or “cyclic amino” is exemplified by the group morpholinyl. The term “alkoxyamino” refers to embodiments wherein at least one of R^{x} is alkoxy. The term “hydroxyamino” refers to embodiments wherein at least one of R^{x} is hydroxy.

“Amino acid” refers to any of the naturally occurring amino acids, as well as synthetic analogs (e.g., D-stereoisomers of the naturally occurring amino acids, such as D-threonine) and derivatives thereof. α-Amino acids comprise a carbon atom to which is bonded an amino group, a carboxyl group, a hydrogen atom, and a distinctive group referred to as a “side chain”. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl or aralkyl (e.g., as in phenylalanine and tryptophan), substituted arylalkyl (e.g., as in tyrosine), and heteroarylalkyl (e.g., as in histidine). The term “naturally occurring amino acids” refers to these amino acids.

Unnatural amino acids are also known in the art, as set forth in, for example, Williams (ed.), Synthesis of Optically Active α-Amino Acids, Pergamon Press (1989); Evans et al., J. Amer. Chem. Soc., 112:4011-4030 (1990); Pu et al., J. Org Chem., 56:1280-1283 (1991); Williams et al., J. Amer. Chem. Soc., 113:9276-9286 (1991); and all references cited therein.

The term “peptide” refers to any of various natural or synthetic compounds containing two or more amino acids linked by the carboxyl group of one amino acid to the amino group of another. A “dipeptide” refers to a peptide that contains 2 amino acids. A “tripeptide” refers to a peptide that contains 3 amino acids. A “tetrapeptide” refers to a peptide that contains 4 amino acids.

The term “aromatic” refers to a cyclic or polycyclic moiety having a conjugated unsaturated (4n+2) π electron system (where n is a positive integer), sometimes referred to as a delocalized π electron system.

The term “aryl” refers to an aromatic cyclic hydrocarbon group of from 6 to 20 carbon atoms having a single ring (e.g., phenyl) or multiple condensed (fused) rings (e.g., naphthyl or anthryl). Aryls include phenyl, naphthyl and the like. The term “aryl” also includes substituted aryl rings and refers to an aryl group as defined above, which unless otherwise constrained by the definition for the aryl substituent, is substituted with one or more, such as 1 to 5, substituents, independently selected from the group consisting of: hydroxy, acyl, acyloxy, alkenyl, alkoxy, alkyl, alkynyl, amino, aryl, aryloxy, azido, carboxyl, alkoxycarbonyl, amido, cyano, cycloalkyl, cycloalkenyl, halogen, heterocyclyl, heterocyclyloxy, nitro, sulfonylamino, aminosulfonyl, sulfanyl, sulfinyl, sulfonyl, and sulfonic acid.

The term “aryloxy” refers to the group —O-aryl.

The term “aralkyl” refers to the group -alkylene-aryl, wherein alkylene and aryl are defined herein.

The term “carbonyl” refers to the di-radical “C═O”, which is also illustrated as “—C(O)—”. This moiety is also referred as “keto.”

The term “alkylcarbonyl” refers to the groups: —C(O)-(alkyl), —C(O)-(cycloalkyl), —C(O)-(alkenyl), and —C(O)-(alkynyl).

The term “alkoxycarbonyl” refers to the groups: —C(O)O-(alkyl), —C(O)O-(cycloalkyl), —C(O)O-(alkenyl), and —C(O)O-(alkynyl). These moieties may also be referred to as esters.

The term “aminosulfonyl” refers to the group —S(O)₂-(amino). The term “sulfonylamino” refers to the group -(amino) —S(O)₂—R^{y}, wherein R^{y} is alkyl, cycloalkyl, alkenyl, aryl or heterocyclyl.

The term “aminocarbonyl” refers to the group —C(O)-(amino) and the term “cabonylamino” refers to the group -amino-C(O)—R^{y}, wherein R^{y} is alkyl, cycloalkyl, alkenyl, aryl or heterocyclyl and the term amino is as described herein.

The term “carboxy” or “carboxyl” refers to the moiety “—C(O)OH,” which is also illustrated as “—COOH.” The salts of —COOH are also included.

The term “cycloalkyl” refers to non-aromatic cyclic hydrocarbon groups having about 3 to 12 carbon atoms having a single ring or multiple condensed or bridged rings. Such cycloalkyl groups include, by way of example, single ring structures such as cyclopropyl, cyclobutyl, cyclopentyl, cyclohexyl, and the like, or multiple ring structures such as adamantyl, and the like. The term “cycloalkyl” additionally encompasses spiro systems wherein the cycloalkyl ring has a carbon ring atom in common with another ring. The term “cycloalkyl” also includes substituted cycloalkyl rings and refers to a cycloalkyl group substituted with one or more, such as 1 to 5, substituents, independently selected from the group consisting of: ═O, ═S, acyl, acyloxy, alkenyl, alkoxy, alkyl, alkynyl, amino, aryl, aryloxy, azido, carboxyl, alkoxycarbonyl, amido, cyano, cycloalkyl, cycloalkenyl, halogen, heterocyclyl, heterocyclyloxy, hydroxyl, nitro, sulfonylamino, aminosulfonyl, sulfanyl, sulfinyl, sulfonyl, and sulfonic acid. A cycloalkyl ring substituted with an alkyl group is also referred as “alkylcycloalkyl.”

The term “cycloalkenyl” refers to cyclic alkenyl groups of from 3 to 10 carbon atoms having single or multiple cyclic rings. This also includes substituted cycloalkenyl which includes substituents as those listed with cycloalkyl.

The term “halo” or “halogen” refers to fluoro, chloro, bromo, and iodo.

The term “heteroaryl” refers to an aromatic carbocyclic radical having one or more, such as 1 to 3, rings incorporating one or more, such as 1 to 4, heteroatoms within the ring (chosen from nitrogen, oxygen, and/or sulfur). This term excludes saturated carbocyclic radical having one or more rings incorporating one or more heteroatoms within the ring (chosen from nitrogen, oxygen, and/or sulfur).

The terms “heterocycle,” “heterocyclic,” “heterocyclo,” and “heterocyclyl” refer to a monovalent, saturated, partially unsaturated or fully unsaturated (aromatic) carbocyclic radical having one or more, such as 1 to 3, rings incorporating one or more, such as 1 to 4, heteroatoms within the ring (chosen from nitrogen, oxygen, and/or sulfur). Heterocycles include morpholine, piperidine, piperazine, thiazole, thiazolidine, isothiazole, oxazole, isoxazole, pyrazole, pyrazolidine, pyrazoline, imidazole, imidazolidine, benzothiazole, pyridine, pyrazine, pyrimidine, pyridazine, pyrrole, pyrrolidine, quinoline, quinazoline, purine, carbazole, benzimidazole, thiophene, benzothiophene, pyran, tetrahydropyran, benzopyran, furan, tetrahydrofuran, indole, indoline, indazole, xanthene, thioxanthene, acridine, quinuclidine, and the like. The terms “heterocycle,” “heterocyclic,” “heterocyclo,” and “heterocyclyl” also include substituted rings and refer to a heterocycle group as defined above, which unless otherwise constrained by the definition for the heterocycle, is substituted with one or more, such as 1 to 5, substituents, independently selected from the group consisting of: hydroxy, acyl, acyloxy, alkenyl, alkoxy, alkyl, alkynyl, amino, aryl, aryloxy, azido, carboxyl, alkoxycarbonyl, amido, cyano, cycloalkyl, cycloalkenyl, halogen, heterocyclyl, heterocyclo-oxy, nitro, sulfonylamino, aminosulfonyl, sulfanyl, sulfinyl, sulfonyl, and sulfonic acid. This term is exemplified by 4,5-dihydroisoxazole-5-methylcarboxylate, 5-butylisoxazol, pyrrolidinyl, morpholinyl, imidazolyl, 5-hydroxypyridin-2-yl, dimethylaminopyridin-3-yl, isoindolinedione, trifluoromethyloxazolyl, 2-bromophenyl-1H-tetrazol-5-yl, methylthiazolyl, phenylthiazolyl, and benzothiazolyl.

The term “heterocyclyloxy” refers to the moiety —O-heterocyclyl.

The term “inflammation,” “inflammatory conditions,” or “inflammation conditions” includes but is not limited to muscle fatigue, osteoarthritis, rheumatoid arthritis, inflammatory bowel syndrome or disorder, Crohn's disease, skin inflammation, such as atopic dermatitis, contact dermatitis, allergic dermatitis, xerosis, eczema, rosacea, seborrhea, psoriasis, atherosclerosis, thermal and radiation burns, acne, oily skin, wrinkles, excessive cellulite, excessive pore size, intrinsic skin aging, photo aging, photo damage, harmful UV damage, keratinization abnormalities, irritation including retinoid induced irritation, hirsutism, alopecia, dyspigmentation, inflammation due to wounds, scarring or stretch marks, loss of elasticity, skin atrophy, and gingivitis.

The term “ischemia” refers to deficiency of blood to an organ or tissue due to functional constriction or actual obstruction of a blood vessel.

The term “isomers” or “stereoisomers” relates to compounds that have identical molecular formulae but that differ in the arrangement of their atoms in space. Stereoisomers that are not mirror images of one another are termed “diastereoisomers” and stereoisomers that are non-superimposable mirror images are termed “enantiomers,” or sometimes optical isomers. A mixture of equal amounts of stereoisomers of a molecule is termed a “racemate” or a “racemic mixture.” A carbon atom bonded to four non-identical substituents is termed a “chiral center.” Certain compounds of the present invention have one or more chiral centers and therefore may exist as either individual stereoisomers or as a mixture of stereoisomers. Configurations of stereoisomers that owe their existence to hindered rotation about double bonds are differentiated by their prefixes cis and trans, (or Z and E), which indicate that the groups are on the same side (cis or Z) or on opposite sides (trans or E) of the double bond in the molecule according to the Cahn-Ingold-Prelog rules. This invention includes all possible stereoisomers as individual stereoisomers, racemates, or mixtures of stereoisomers.

A “lipoxygenase-mediated condition” or a “disorder mediated by lipoxygenases” means any condition, disorder or disease mediated, at least in part, by a lipoxygenase enzyme. This includes disorders related to or otherwise associated with a lipoxygenase enzyme or the inhibition thereof, including, by way of example and without limitation, diseases involving apoptosis in cancer cells such as prostatic cancer, gastric cancer, breast cancer, pancreatic cancer, colorectal or esophageal cancer and airways carcinoma; diseases involving hypoxia, or anoxia such as atherosclerosis, myocardial infarction, cardiovascular disease, heart failure (including chronic and congestive heart failure), cerebral ischemia, retinal ischemia, myocardial ischemia, post surgical cognitive dysfunction and other ischemias; diseases involving inflammation, including diabetes, arterial inflammation, inflammatory bowel disease, Crohn's disease, renal disease, pre-menstrual syndrome, asthma, allergic rhinitis, gout; cardiopulmonary inflammation, rheumatoid arthritis, osteoarthritis, muscle fatigue and inflammatory disorders of the skin including acne, dermatitis and psoriasis; disorders of the airways such as asthma, chronic bronchitis, human airway carcinomas, mucus hypersecretion, chronic obstructive pulmonary disease (COPD), pulmonary fibrosis caused by chemotherapy or other drugs, idiopathic pulmonary fibrosis, cystic fibrosis, and adult respiratory distress syndrome; diseases involving central nervous system (CNS) disorders including psychiatric disorders including anxiety and depression; neurodegeneration and neuroinflammation including Alzheimer's, dementia and Parkinson's disease; peripheral neuropathy including spinal chord injury, head injury and surgical trauma, and allograft tissue and organ transplant rejection; diseases involving the autoimmune system such as psoriasis, eczema, rheumatoid arthritis, and diabetes; and disorders involving bone loss or bone formation.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to salts which retain the biological effectiveness and properties of the compounds of this invention and which are not biologically or otherwise undesirable. In some cases, the compounds of this invention are capable of forming acid and/or base salts by virtue of the presence of phenolic, amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl)amines, tri(substituted alkyl)amines, alkenyl amines, dialkenyl amines, trialkenyl amines, substituted alkenyl amines, di(substituted alkenyl)amines, tri(substituted alkenyl)amines, cycloalkyl amines, di(cycloalkyl)amines, tri(cycloalkyl)amines, substituted cycloalkyl amines, disubstituted cycloalkyl amine, trisubstituted cycloalkyl amines, cycloalkenyl amines, di(cycloalkenyl)amines, tri(cycloalkenyl)amines, substituted cycloalkenyl amines, disubstituted cycloalkenyl amine, trisubstituted cycloalkenyl amines, aryl amines, diaryl amines, triaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heterocyclic, and the like. Also included are amines where the two or three substituents, together with the amino nitrogen, form a heterocyclic group.

Specific examples of suitable amines include, by way of example only, isopropylamine, trimethyl amine, diethyl amine, tri(iso-propyl)amine, tri(n-propyl)amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

It should be understood that for the purpose of this invention, all references to acceptable salts also include solvent addition forms (solvates) or polymorphs (crystal forms). “Solvate” means solvent addition form that contains either stoichiometric or non-stoichiometric amounts of solvent. Some compounds have a tendency to trap a fixed molar ratio of solvent molecules in the crystalline solid state, thus forming a solvate. If the solvent is water the solvate formed is a “hydrate,” when the solvent is alcohol, the solvate formed is an “alcoholate.” “Polymorphs” (or “crystal forms”) means crystal structures in which a compound can crystallize in different crystal packing arrangements, all of which have the same elemental composition. Different crystal forms usually have different X-ray diffraction patterns, infrared spectra, melting points, density, hardness, crystal shape, optical and electrical properties, stability and solubility. Recrystallization solvent, rate of crystallization, storage temperature, and other factors may cause one crystal form to dominate.

The term “prodrug” refers to an inactive form of a compound which must be metabolized in vivo, e.g., by biological fluids or enzymes, by a subject after administration into an active form of the parent compound in order to produce the desired pharmacological effect. The prodrug can be metabolized before absorption, during absorption, after absorption, or at a specific site. Prodrug forms of compounds may be utilized, for example, to improve bioavailability, improve subject acceptability such as masking or reducing unpleasant characteristics such as a bitter taste, odor, or gastrointestinal irritability, alter solubility, provide for prolonged or sustained release or delivery, improve ease of formulation, or provide site-specific delivery of the compound.

Prodrugs of a compound of this invention are prepared by modifying one or more functional group(s) present in the compound in such a way that the modification(s) may be cleaved in vivo to release the parent compound. Prodrugs include compounds wherein a hydroxyl group in a compound of the invention is bonded to any group that may be cleaved in vivo to regenerate the free hydroxyl, amino. Examples of prodrugs include, but are not limited to, esters (e.g., acetate, formate, and benzoate derivatives), carbamates (e.g., N,N-dimethylaminocarbonyl) of hydroxy functional groups in compounds of the invention, see Bundegaard, H. Design of Prodrugs. New York-Oxford: Elsevier, 1985, pp. 1-92, and the like. Reference to a compound herein includes prodrug forms of said compound.

The term “subject” includes, but is not limited to, humans and animals, such as farm animals (cattle, horses, sheep, goats, and swine) and domestic animals (rabbits, dogs, cats, rats, mice and guinea pigs. The term “subject” does not denote a particular age or sex.

The term “sulfanyl” or “thio” refers to the groups: —S—H, —S-(alkyl), —S-(aryl), or —S-(heterocyclyl). The term is exemplified by groups such as isopropylthio and methyl thioacetate.

The term “pharmaceutical” refers to an agent or mixture of agents that is primarily intended to treat or ameliorate a disease or disorder. A pharmaceutical may be available only by prescription or may be available “over-the-counter” (OTC); in either case, its formulation and distribution are generally regulated by a governmental authority charged with such regulation, such as the Food and Drug Administration (FDA) in the United States.

The term “pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

The term “pharmaceutically effective amount” or “therapeutically effective amount” refers to that amount of a compound of this invention that is sufficient to effect treatment, as defined below, when administered to a subject in need of such treatment. The therapeutically effective amount will vary depending upon the subject and disease condition being treated, the weight and age of the subject, the severity of the disease condition, the particular compound chosen, the dosing regimen to be followed, timing of administration, the manner of administration and the like, all of which can readily be determined by one of ordinary skill in the art.

The term “treatment” or “treating” means any treatment of a disease or disorder in a subject, including:

preventing or protecting against the disease or disorder, that is, causing the clinical symptoms not to develop;

inhibiting the disease or disorder, that is, arresting or suppressing the development of clinical symptoms; and/or

relieving the disease or disorder that is, causing the regression of clinical symptoms.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

The term “antioxidants” include, but are not limited to, water-soluble antioxidants such as sulfhydryl compounds and their derivatives (e.g., sodium sulfite, sodium bisulfite, sodium metabisulfite, sodium thiosulfite, sodium formaldehyde sulfoxylate, thioglycerol, thiosorbitol, thiourea, thioglycolic acid, cysteine hydrochloride, N-acetyl-cysteine, and mixtures thereof), lipoic acid and dihydrolipoic acid, resveratrol, lactoferrin, ascorbic acid and ascorbic acid derivatives (e.g., ascorbyl palmitate and ascorbyl polypeptide). Oil-soluble antioxidants suitable for use in the formulation include, but are not limited to, hydroquinones (i.e. ubiquinone, coenzyme Q10), propyl gallate, and nordihydroguiaretic acid. Natural extracts containing antioxidants suitable for use in the formulations of this invention include, but are not limited to, extracts containing flavonoids and isoflavonoids and their derivatives (e.g., genistein and diadzein), extracts containing polyphenols (i.e. resveratrol and catechins), and the like. Examples of such natural extracts include grape seed, green tea, pine bark, and propolis. Other examples of antioxidants may be found on pages 1612-13 of Wenninger, J. A. and G. N. McEwen, International Cosmetic Ingredients Dictionary and Handbook, Seventh Edition, The Cosmetic, Toiletry, and Fragrance Assoc., Washington, D.C., 1997 (hereinafter referred to as “ICI Handbook”). Other known antioxidants compatible with the other components of the compositions are contemplated in the invention.

The term “cosmetic agents” includes compounds that have a cosmetic or therapeutic effect on the skin, hair, or nails, e.g., lightening agents, darkening agents such as self-tanning agents, anti-acne agents, shine control agents, anti-microbial agents, anti-inflammatory agents, anti-mycotic agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, firming agents, anti-callous agents, and agents for hair, nail, and/or skin conditioning. Examples of cosmetic agents are hydroxy acids, benzoyl peroxide, sulfur resorcinol, ascorbic acid, D-panthenol, hydroquinone, octyl methoxycinnamate, titanium dioxide, octyl salicylate, homosalate, avobenzone, polyphenols, carotenoids, free radical scavengers, spin traps, retinoids such as retinol and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, peptides such as those disclosed in WO 2000/15188, lipoic acid, amino acids such a proline and tyrosine, vitamins, lactobionic acid, acetyl-coenzyme A, niacin, riboflavin, thiamin, ribose, electron transporters such as NADH and FADH2, and other botanical extracts such as aloe vera and soy extracts, and derivatives and mixtures thereof. The cosmetic agent will typically be present in the formulation of the invention in an amount of from about 0.001% to about 20% by weight of the formulation, e.g., about 0.01% to about 10% such as about 0.1% to about 5%.

The term “cosmetics” includes make-up, foundation, and skin care products. The term “make-up” refers to products that leave color on the face, including foundations, mascara, concealers, eye liners, brow colors, eye shadows, blushers, lip colors, and so forth. The term “foundation” refers to liquid, cream, mousse, pancake, compact, or like products that even out the overall coloring of the skin. Foundation is typically manufactured to work better over moisturized and/or oiled skin.

As used herein, “cosmetically-acceptable” means that the product(s), ingredient(s), or compound(s) which the term describes are suitable for use in contact with skin without undue toxicity, incompatibility, instability, irritation, allergic response, or the like. This term is not intended to limit the ingredient/product to which it describes for use solely as a cosmetic product (e.g., the ingredient may be used as a prescription or over-the-counter pharmaceutical product).

As used herein, “cosmetically acceptable carrier” includes any and all solvents, dispersion media, coatings, antibacterial and antifungal agents, isotonic and absorption delaying agents, and the like. The use of such media and agents for dermatologically active substances is well known in the art. Except insofar as any conventional media or agent is incompatible with the active ingredient, its use in the therapeutic compositions is contemplated. Supplementary active ingredients can also be incorporated into compositions of the invention.

The terms “cosmetically-acceptable organic solvent” and “pharmaceutically-acceptable organic solvent” refer to a solvent which is capable of having a composition of the present invention dispersed or dissolved therein, and of possessing acceptable safety properties (e.g., irritation and sensitization characteristics). Examples of suitable organic solvents include propylene glycol, polyethylene glycol (200-600), polypropylene glycol (425-2025), glycerol, 1,2,4-butanetriol, sorbitol esters, 1,2,6-hexanetriol, ethanol, isopropanol, butanetriol, sorbitol esters, 1,2,6-hexanetriol, ethanol, isopropanol, butanediol, and mixtures thereof.

The term “retinoid” as used herein refers to retinol (vitamin A alcohol), retinal (vitamin A aldehyde), retinoic acid (vitamin A acid), and C₂-C₂₀ retinyl esters of retinol or mixtures thereof. The term “retinol” includes the following isomers of retinol: all-trans-retinol, 13-cis-retinol, 11-cis-retinol, 9-cis-retinol, and 3,4-didehydro-retinol. Exemplary isomers are all-trans-retinol, 13-cis-retinol, 3,4-didehydro-retinol, 9-cis-retinol. Due to its wide commercial availability, all-trans-retinol is most often used. Retinyl ester is an ester of retinol. Examples of retinyl esters include: retinyl palmitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl laurate, retinyl tridecanoate, retinyl myristate, retinyl pentadecanoate, retinyl heptadeconoate, retinyl stearate, retinyl isostearate, retinyl nonadecanoate, retinyl arachidonate, retinyl behenate, retinyl linoleate, retinyl oleate, retinyl lactate, retinyl glycolate, retinyl hydroxy caprylate, retinyl hydroxy laurate, retinyl tartarate. Included in the term “retinoic acid” are 13-cis retinoic acid and all-trans retinoic acid. Another well known retinol is Tretinoin®, better known as Retin-A®. Other brand names include Isotretinoin®, Avita® Cream, and Renova®. Compositions of the present invention may additionally include a retinoid.

The term “skin care composition” or “skin care products” refers to a formulation that includes active ingredients such as the compositions of the present invention, formulated for use in providing beneficial effects to the skin. Skin care compositions include, but are not limited to, skin care products, pharmaceutical products and cosmetics, and may be formulated as topical, transdermal, or oral compositions. The term “skin care products” refers to products used to treat or otherwise care for, moisturize, improve the appearance or feel of, or clean the skin or scalp. Included in this term are products used to treat, or otherwise care for or clean the scalp. Exemplary products covered by the phrase “skin care products” include, but are not limited to, moisturizers, acne treatments, antiperspirants, clarifiers, exfoliators, firming/cellulite treatments, lip products (moisturizers, balms and protectants), masks, oil/shine control, pore strips, shave preparations, skin lighteners, skin lifters, anti-wrinkle treatments, toners, solid emulsion compact, after-shave preparations, shampoos, conditioners, and the like. The term “skin care products” may include, but is not limited to, skin protectant active ingredients, astringent active ingredients, external analgesic, anesthetic and antipruritic active ingredients as published in 21 CFR 347.10, 347.12 and 348.10, and other ingredients as published in 55 CFR 3370, or mixtures thererof. The term “external analgesic, anesthetic, and antipruritic active ingredients” includes, but is not limited to, benzocaine, butamaben picrate, dibucaine, dibucaine hydrochloride, dimethiosoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium resorcinol, tripelennamine hydrochloride, aspirin, hydrocortisone, hydrocortisone acetate, and diphenydramine hydrochloride.

The term “skin protectant active ingredients” include, but are not limited to allantoin, aluminum hydroxide gel, calamine, cocoa butter, cod liver oil, colloidal oatmeal, dimethicone, glycerin, hard fat, kaolin, lanolin, mineral oil, petrolatum, soy products, sodium bicarbonate, topical starch, white petrolatum, zinc acetate, and zinc oxide. Skin protectant active ingredients may also include sunscreen agents.

The term “soy product” or “soy extract” is a substance derived from the soybean, containing the ingredients naturally found in soybeans, at the relative concentrations as found in the beans. In some embodiments, the soy product is a non-denatured soy product. The latter is a soy product, which has been obtained by processes that leave the active proteins intact by carefully controlling the process parameters such as the temperature, the extraction media. This can be measured, for example, by the presence of intact soybean trypsin inhibitor (STI) protein. The soy products that can be used may be in the form of a fluid (e.g., soymilk) or a solid (e.g., a soybean powder or soymilk powder). Compositions of the present invention may additionally include a soy product or soy extract.

The term “sunscreen” may include, but is not limited to, organic or inorganic sunscreens, sun blocks titanium oxide and zinc oxide, and skin protectants and/or mixtures thereof. Sunscreen products providing a minimum SPF value of not less than 2 include, but are not limited to, aminobenzoic acid (PABA), avobenzone, cinoxate, dioxybenzone, homosalate, methyl anthranilate, methoxycinnamate, octocrylene, octyl methoxycinnamate, octyl salicylate, oxybenzone, padimate O, phenylbenzimidazole sulfonic acid, sulisobenzone, titanium dioxide, trolamine salicylate, titanium oxide, and zinc oxide.

The term “tocopherol” means alpha-, beta-, gamma-, or delta-tocopherol. This term includes mixed tocopherols from edible vegetable oils sold commercially, for example by Cargill Health and Food Technologies (Minneapolis, Minn.); by Archer Daniels Midland Company (ADM, Decatur, Ill.) under the name Decanox® MTS-50, Decanox® MTS-70 and Decanox® MTS-90; or by Cognis Nutrition & Health (www.cognis.com) under the name Covi-ox® T-50, Covi-ox® T-70, and Covi-ox® T-90. Cargill Health and Food Technologies mixed tocopherols contain 50-70% gamma-tocopherol, 15-30% delta-tocopherol, <5% beta-tocopherol, and <20% alpha-tocopherol, and Covi-ox® typically contains 60% gamma-tocopherol, 24%-delta-tocopherol, 2% beta-tocopherol and 14% alpha-tocopherol. Mixed tocopherol formulations including alpha- beta-, delta-, and gamma-tocopherols as well as Vitamin E (essentially alpha tocopherol) are sold as OTC dietary supplements; accordingly, these ingredients may be used in oral, as well as topical and transdermal formulations of the present invention.

The term “topical application” means directly laying on or spreading on outer skin using, e.g., by use of the hands or an applicator such as a wipe, puff, roller, or spray. As used herein, “topical carrier” means one or more compatible solid or liquid filler diluents that are suitable for topical administration to a mammal. Examples of topical carriers include, but are not limited to, water, waxes, oils, emollients, emulsifiers, thickening agents, gelling agents, and mixtures thereof.

The term “transdermal application” generally refers to methods in which a composition is delivered selectively to one area or “patch” of skin by a special applicator that is designed to contact an area of the skin and continuously deliver compound to that area for a period time.

The term “vitamins” include, but are not limited to, vitamin A, vitamin Bs such as vitamin B3, vitamin B5, and vitamin B12, vitamin C, vitamin K, vitamin E, tocopherols and derivatives thereof.

Nomenclature

In general, the nomenclature used in this Application was generated using or with the help of the naming package within the ChemDrawUltra® version 9.0.1 suite of programs by CambridgeSoft Corp. (Cambridge, Mass.).

### Synthesis of the Compounds of the Invention

Synthetic Reaction Parameters

The terms “solvent,” “inert organic solvent” or “inert solvent” mean a solvent inert under the conditions of the reaction being described in conjunction therewith. Solvents employed in synthesis of the compounds of the invention include, for example, methanol (“MeOH”), acetone, water, acetonitrile, 1,4-dioxane, dimethylformamide (“DMF”), benzene, toluene, tetrahydrofuran (“THF”), chloroform, methylene chloride (also named dichloromethane (“DCM”)), diethyl ether, ethyl acetate (“EtOAc”), pyridine and the like, as well as mixtures thereof. Unless specified to the contrary, the solvents used in the reactions of the present invention are inert organic solvents.

The term “q.s.” means adding a quantity sufficient to achieve a stated function, e.g., to bring a solution to the desired volume (i.e., 100%), and “MOM” refers to methoxymethyl.

Unless specified to the contrary, the reactions described herein take place at atmospheric pressure within a temperature range from −10° C. to 110° C. and in some cases at “room” or “ambient” temperature, e.g., 20° C. Further, unless otherwise specified, the reaction times and conditions are intended to be approximate.

Isolation and purification of the compounds and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

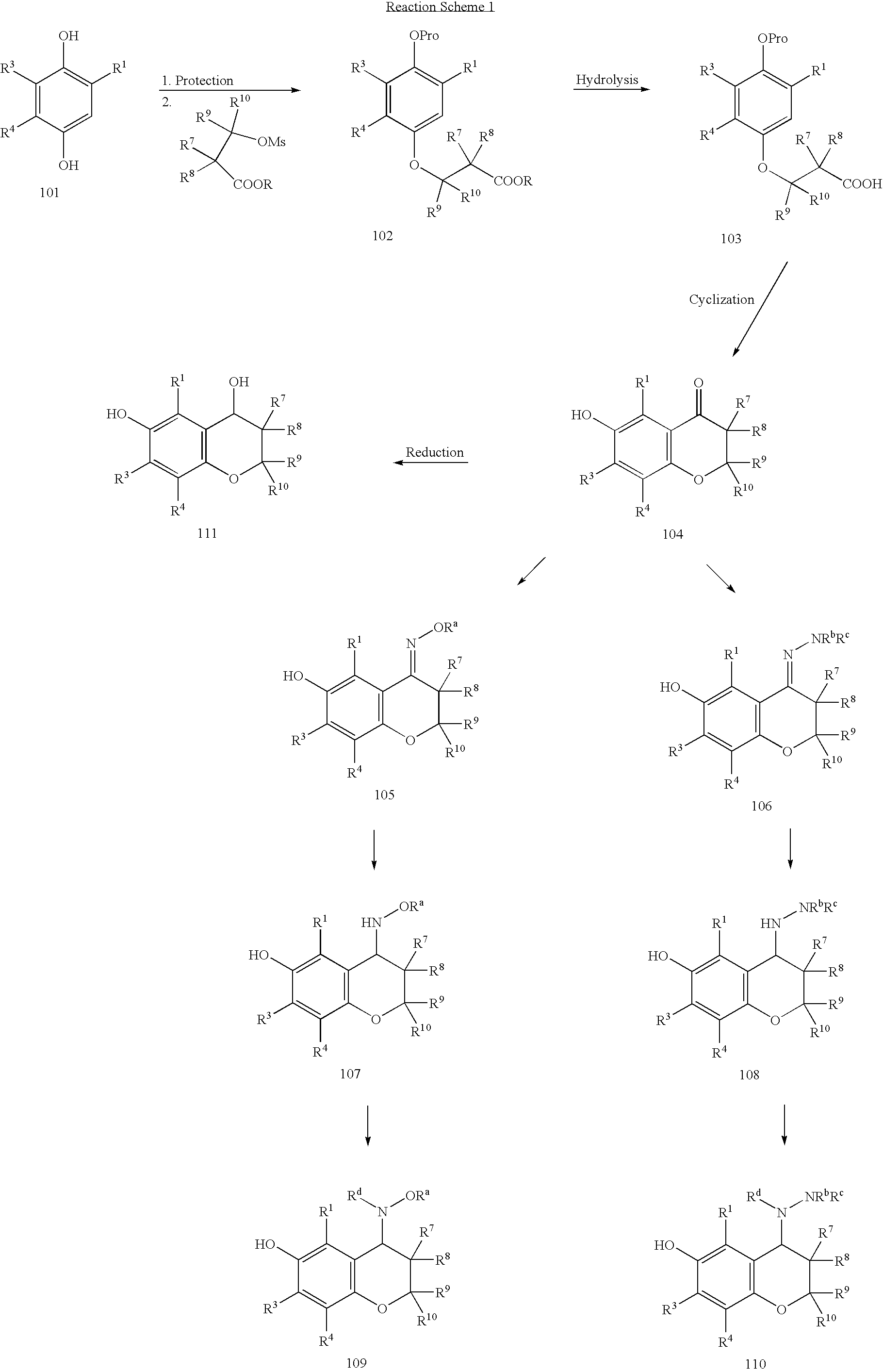

Scheme 1 describes a synthesis for compounds of Formula I, wherein X is O, and R⁵ and R⁶ together form a C═NOR^{a} or a C═—NR^{b}R^{c} or R⁵ is —NR^{d}OR^{a} or —NR^{d}—NR^{b}R^{c} and R⁶ is hydrogen, and R, R¹, R³, R⁴, R⁷, R⁸, R⁹ and R¹⁰ are as defined above. One of the hydroxyl groups of the hydroquinone of Formula 101 is protected with, for example, a benzyl group, by reaction with one equivalent of for example benzyl bromide. Addition of 1-methanesulfonyloxymethyl-carboxylic acid ester to the protected hydroquinone in a solvent such as dimethylformamide in the presence of a base such as cesium carbonate, may yield a compound of Formula 102, wherein R is alkyl, which after hydrolysis and cyclization may yield the 4-chromanone derivative of Formula 104. Addition of hydroxylamine or alkoxyamine hydrochloride may result in the oxime of Formula 105, wherein R^{a} is hydrogen or alkyl respectively. The oxime can be reduced to hydroxylamines or alkoxyamines of Formula 107 by simple addition of hydrogen which can be accomplished with borane in a solvent such as tetrahydrofuran or pyridine, or with sodium cyano borohydride. Similarly, condensation of a hydrazine to the keto group of compound of Formula 104, may yield the hydrazones of Formula 106, which may be reduced to hydrazines of Formula 108.

The hydroxylamines of Formula 107 or the hydrazines of Formula 108 may be further alkylated with a halo alkane or with an aldehyde followed by reductive amination to yield the alkylated compounds of Formula 109 and Formula 110, respectively. The 4-chromanone derivative of Formula 104 may also be reduced with for example sodium borohydride to yield the 4,6-dihydroxy derivative of Formula 111.

This scheme may also be used for the preparation of thiochromans of this invention by substituting the hydroquinone of Formula 101 with the corresponding 4-mercaptophenol.

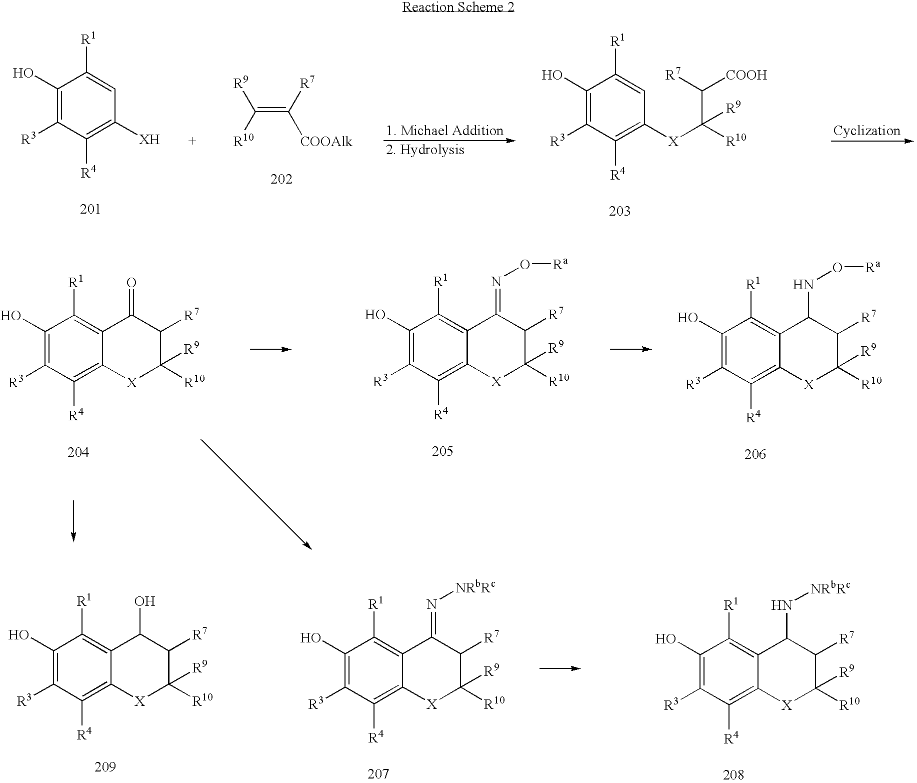

Scheme 2 describes a synthesis for compounds of Formula I of the present invention wherein R⁵ and R⁶ independently of each other are —NOR^{a}, —NH—NR^{b}R^{c}; or OH or together with the carbon atom to which they are attached form a C═NOR^{a} or a C═N—NR^{b}R^{c} group, R⁸ is hydrogen, and X, R¹, R³, R⁴, R⁷, R⁹, R¹⁰, R^{a}, R^{b}, and R^{c} are as defined above. Under Michael addition conditions, the phenol of Formula 201 is condensed with an acrylate of Formula 202, wherein Alk is an alkyl group, in an anhydrous solvent such as alkanol, for example methanol or ethanol, and the presence of a strong base such as sulfuric acid. The obtained ester is hydrolyzed in the presence of a base such as sodium or potassium hydroxide to give the acid of Formula 203, which can be cyclized under acidic conditions to give the 4-keto compound of Formula 204. Addition of hydroxylamine or alkoxyamine hydrochloride may yield an oxime of Formula 205 that can be reduced with, for example, sodium cyano borohydride or borane/pyridine to give the alkoxyamine of Formula 206. Similarly, addition of hydrazine may yield the hydrazone derivative of Formula 207 that may be similarly reduced to yield the hydrazine of Formula 208. As described in Scheme 1, the compound of Formula 204 may be further reduced with, for example, sodium borohydride to form the compound of Formula 209.

### Preferred Compounds

The compounds of Formula I encompass the derivatives of the invention as disclosed, and/or the pharmaceutically acceptable salts of such compounds. In addition, the compounds of this invention include the individual stereochemical isomers and mixtures thereof, arising from the selection of substituent groups. It will be understood by those skilled in the art with respect to any group containing one or more substituents that such groups are not intended to introduce any substitution or substitution patterns that are sterically impractical and/or synthetically non-feasible.

### Utility, Testing and Administration

General Utility

Without subscribing to a particular theory or mechanism of action, compounds of the invention may target certain enzymes known as “oxidoreductases” that function widely across a variety of physiological processes, for example, certain compounds of the present invention may target lipoxygenases such as 5-Lipoxygenase, 12-Lipoxygenase, 15-Lipoxygenase, and/or 12/15-Lipoxygenase. In particular, oxidoreductases catalyze reactions in which two molecules interact so that one molecule is oxidized and the other is reduced. Alterations in oxidoreductases are thought to account for as many as 3% of all known human genetic diseases. Abnormalities in oxidoreductase activity may underlie such disorders as congestive heart failure, respiratory chain defects (e.g., abnormalities associated with enzymes of the respiratory chain, acute respiratory distress syndrome (ARDS)), glycogen storage disease, end-stage renal disease, and rheumatoid arthritis. Inhibitors of lipoxygenases are known to be useful in the prevention or treatment of, for example, disorders selected from apoptosis in cancer cells including prostatic cancer, gastric cancer, breast cancer, pancreatic cancer, colorectal or esophageal cancer and airways carcinoma; diseases involving hypoxia or anoxia, including atherosclerosis, myocardial infarction, cardiovascular disease, heart failure (including chronic and congestive heart failure), cerebral ischemia, retinal ischemia, myocardial ischemia, post surgical cognitive dysfunction and other ischemias; diseases involving inflammation, including diabetes, arterial inflammation, inflammatory bowel disease, Crohn's disease, renal disease, pre-menstrual syndrome, asthma, allergic rhinitis, gout, cardiopulmonary inflammation, rheumatoid arthritis, osteoarthritis, muscle fatigue and inflammatory disorders of the skin including acne, dermatitis and psoriasis; disorders of the airways including asthma, chronic bronchitis, human airway carcinomas, mucus hypersecretion, chronic obstructive pulmonary disease (COPD), pulmonary fibrosis caused by chemotherapy or other drugs, idiopathic pulmonary fibrosis, cystic fibrosis, and adult respiratory distress syndrome; diseases involving central nervous system (CNS) disorders including psychiatric disorders including anxiety and depression; neurodegeneration and neuroinflammation including Alzheimer's, dementia and Parkinson's disease; peripheral neuropathy including spinal chord injury, head injury and surgical trauma, and allograft tissue and organ transplant rejection; diseases involving the autoimmune system including psoriasis, eczema, rheumatoid arthritis, and diabetes; and disorders involving bone loss or bone formation

Certain compounds of the present invention are also useful in skin care compositions for treating conditions falling with the group of dermatologic conditions, such as prevention and protection of skin tissue against age-related damage or damage resulting from insults such as harmful ultraviolet (UV) radiation, use of retinoids, wearing diapers, stress and fatigue, and in the treatment of contact dermatitis, skin irritation, skin pigmentation, psoriasis, or acne.

Compositions in accordance with the present invention may be used either alone or as part of topical and transdermal formulations for a number of indications, such as for maintaining or improving the appearance of healthy tone, color and body of skin, by reducing or maintaining the production of sebum as further described herein.

Topical agents in accordance with the present invention (creams, serums, lotions, ointments, liniments, and the like), may be utilized for reducing or maintaining oily skin, reducing or maintaining sebum production and reducing or maintaining disorders resulting from oily skin or sebum overproduction, as well as treating or ameliorating skin discomforts resulting from such conditions.

Generally, formulations of compositions of the invention may be used to provide inhibition or regulation of sebum production, to bring sebum homeostasis to a normal level, to remove sebum from the skin, to inhibit or treat oily skin, to prevent or inhibit the development of acne, to treat acne when present, to reduce or inhibit comedone formation, and to remove or clear comedones.

Assessment of efficacy of a particular formulation may be made in one or more pre-clinical or clinical assays known in the art, including, but not limited to, the in vitro sebocyte lipogenesis assay, the healthy skin assessment/improvement assay. Appropriate pre-clinical assays are detailed in the Examples section.

Compositions of the present invention may be used in cosmetic compositions and skin care products. Cosmetic compositions of the present invention are ideally suited for use in treating the skin and scalp. The compositions are useful in preparations having as their primary goal conditioning for the skin, improved skin feel and appearance, regulating skin texture, or the like, by, inter alia, regulating or reducing the sebum overproduction and/or disorders resulting thereof such as acne, comedone formation, and oily skin. The compositions are also useful in preparations having as their primary goal moisturizing and/or conditioning for the hair, improved feel, regulating texture and luster, or the like, by regulating or reducing the sebum overproduction on the scalp.

The compositions of the invention can accordingly be applied to the skin and/or scalp in the traditional manner with or without a conventional holder or applicator to provide a decorative and/or protective film thereto. Cosmetics include make-up, such as foundations, mascara, concealers, eye liners, brow colors, eye shadows, blushers, lip colors, and so forth.

Skin care products are products that are used to treat or otherwise care for, improve the appearance or feel of, or clear the skin of irregularities and comedones. Skin care products include, but are not limited to, acne-care, after-shave preparations, moisturizers, acne treatments, clarifiers, exfoliators, firming/cellulite treatments, lip products (moisturizers, balms and protectants), masks, oil/shine control, pore strips, shave preparations, skin lighteners, tissues, toners, wipes, solid emulsion compact, shampoos, conditioners, and the like.

By way of further example, compositions and methods of the present invention may be useful in treating acne, a skin condition characterized by an inflammatory component arising from the obstruction of the pores and formation of comedones and blackheads caused by sebum overproduction. By way of further example, compositions and methods of the present invention may be useful in the reduction of the appearance of oily skin and hair associated with sebum overproduction.

For certain compositions a useful active ingredient in these compositions is one or more skin protectant active ingredients or emollients. As used herein, the term “emollient” is a material that softens, soothes, supples, coats, lubricates, and/or moisturizes the skin.

Testing

This section describes how compositions incorporating compositions of the present invention are selected, using in vitro and/or in vivo models, and used as therapeutic interventions in the exemplary indications in support of the present invention.

The 5-Lipoxygenase pathway is a major synthetic pathway relevant to human inflammatory disease. The enzyme 5-Lipoxygenase catalyses the two first steps in the oxygenation of arachidonic acid (a polyunsaturated 20-carbon fatty acid) to leukotrienes. Leukotrienes are known to be important mediators of inflammatory and allergic reactions. The first step in the synthesis of leukotrienes, which is catalyzed by 5-Lipoxygenase, is the formation of 5-HPETE. The rearrangement of 5-HPETE to form the unstable LTA₄, the rate-limiting step in the synthesis of the leukotrienes, is also catalyzed by 5-Lipoxygenase. LTA₄ is then converted to either LTB₄ or LTC₄. LTC₄ is rapidly metabolized to LTD₄ and then to LTE₄. LTC₄, LTD₄ and LTE₄ are collectively referred to as the cysteinyl (Cys) leukotrienes.

Biosynthesis of LTB₄, LTC₄, LTD₄ and LTE₄ occurs predominantly in leukocytes, in response to a variety of immunological stimuli. The primary target of LTB₄ is the leukocyte where it elicits enzyme release, chemotaxis, adherence, and aggregation in nM concentrations. LTB₄ modulates immune responses and participates in the host-defense against infections. Hence, LTB₄ is an important chemical mediator in the development and maintenance of inflammatory reactions and disease states.

Endogenous lipoxygenase metabolites may also be involved in enhanced cytokine tumor necrosis factor α (TNF-α) production following certain stimuli such as silica, asbestos and lipopolysaccharides (Rola-Pleszczynski, M et al. Mediators of Inflammation 1: 5-8 (1992)). Consistent with selective lipoxygenase inhibitory effect, certain compounds of the present invention have also shown to have an inhibitory effect on TNF-α. synthesis and/or release. The “TNF-α” has a broad spectrum of biological activities, plays an important role in coordinating the body's response to infection, and serves as an important mediator of inflammation. It is known that inflammatory cytokines have been shown to be pathogenic in several diseases including, but not limited to asthma (N. M. Cembrzynska et al., Am. Rev. Respir. Dis., 147, 291 (1993)), Adult Respiratory Distress Syndrome (ARDS). (Miller et al., Lancet 2 (8665); 712-714 (1989) and Ferrai-Baliviera et al., Arch. Surg. 124 (12): 1400-1405 (1989)), lung fibrosis (Piguet et al., Nature, 344:245-247 (1990) and Bissonnette et al., Inflammation 13 (3): 329-339 (1989)), bone resorption diseases (Bertolini et al., Nature 319: 516-518 (1986) and Johnson et al., Endocrinology 124 (3): 1424-1427 (1989)), auto-immune diseases (W. Fiers, FEBS Lett., 1991, 285, p. 199). It will be therefore appreciated that compounds of the present invention showing an inhibitory effect on both 5-Lipoxygenase and TNF-α should be superior in the treatment or amelioration of for example diseases such as respiratory disorders, antiprolilferative disorders or autoimmune disorders.

In vitro evaluation of the ability of a composition to inhibit the enzymes 5-Lipoxygenase, 15-Lipoxygenase, or 12/15-Lipoxygenase as described in Walidge, N. B. et al. Anal. Biochem., Vol. 231 (1995), pp. 354-358 using a high throughput colorimetric method; as well as in vitro evaluation of inhibiting LTB₄ is described in Examples.

In vitro cell-based assays for inflammation are well known in the art, for example, e-selectin (also named Endothelial Leukocyte Adhesion Molecule or ELAM) or C-reactive protein (CRP). The ELAM assay measures in vitro activity of the test compounds in reducing expression of ELAM in activated endothelial cells. Briefly, endothelial cells are created by adding known activators such as lipopolysaccharides, TNF or IL-1β, alone or in some combination. Activated cells produce ELAM, which can be measured using, for example, an E-selectin monoclonal antibody-based ELISA assay.

In vivo evaluation of anti-inflammatory activity can be determined by well characterized assays measuring Carrageenan-Induced Paw Edema, by Mouse Ear Inflammatory Response to Topical Arachidonic Acid (Gabor, M. Mouse Ear Inflammation Models and their Pharmacological Applications (2000)), or by the in vivo murine Zymosan peritonitis assay. Carrageenan-Induced Paw Edema is a model of inflammation, which causes time-dependent edema formation following carrageenan administration into the intraplantar surface of a rat paw. The application of arachidonic acid (AA) to the ears of mice produces immediate vasodilation and erythema, followed by the abrupt development of edema, which is maximal at 40 to 60 min. The onset of edema coincides with the extravasations of protein and leukocytes. After one hour the edema wanes rapidly and the inflammatory cells leave the tissue so that at 6 hours the ears have returned to near normal.

Administration of Zymosan-A, a purified polysaccharide fraction of yeast cell wall has been used since the 1980s to induce acute inflammatory response in rodents. The inflammatory response is characterized by marked induction of pro-inflammatory cytokines, influx of inflammatory cells and biosynthesis of arachidonic acid metabolites as early as five minutes after the Zymosan injection. The purpose of this model is to evaluate the ability of compounds to reduce inflammatory response induced by administration of Zymosan-A and assessed by the level of inflammatory cytokines and arachidonic metabolites in the fluid exudates.

These assays, as described in the Examples, measure a test compound's ability to treat these inflammatory processes via systemic and topical routes of administration.

Protection against redox stress can be evaluated in cell culture using high glutamate induced oxidative stress (HGOS) in mouse dopaminergic cell lines. The cytotoxic effect of glutamate is not due to excitotoxicity, as this cell line is devoid of inotropic glutamate receptors. Rather, the glutamate-induced toxicity of dopaminergic cells is associated with an inhibition of cystine transport which subsequently leads to depletion of intracellular glutathione (GSH) levels (Murphy T. H., et al. Neuron, Vol. 2 (1989), pp. 1547-1558), activation of neuronal 12-Lipoxygenase (Li, Y. et al. Neuron, Vol. 19 (1997), pp. 453-463), increased ROS production (Tan S. et al. J. Cell Biol., Vol. 141 (1998), pp. 1423-1432) and elevated intracellular Ca²⁺ (Li, Y. et al. see supra). Some molecules were measured for their ability to protect cells against glutamate-induced stress and the assay is detailed in Examples.

Further validation of neuroantiinflammatory activity of compounds can be assessed in vitro by the inhibition of IL-1.beta. release from a microglial cell line.

Interleukin-1 (IL-1) is a pro-inflammatory cytokine that exists in two separate forms that share 30% sequence homology (alpha and beta). Constitutive expression of IL-1 is low in the brain but levels of both forms of this cytokine increase dramatically after injury. There is substantial evidence that IL-1 is an important mediator of neurodegeneration induced by cerebral ischemia (Touzani, O. et al. J. Neuroimmunol., Vol. 100 (1999), pp. 203-215). Both IL-1 forms are rapidly induced in experimental models of stroke and administration of recombinant IL-1β enhances ischemic injury (see Hill J. K., et al. Brain Res., Vol. 820 (1999), pp. 45-54); Hillhouse E. W. et al. Neurosci. Lett. Vol. 249 (1998), pp. 177-179; Loddick S. A. et al. J. Cereb. Blood Flow Metab. Vol. 16 (1996), pp.:932-940; Stroemer R. P. et al. J. Cereb. Blood Flow Metab. Vol. 18 (1998), pp. 833-839). Conversely, blocking IL-1 actions with a receptor antagonist or a neutralizing antibody markedly reduces neuronal death and inflammation in models of ischemic damage (see Betz, A. L., J. Cereb. Blood Flow Metab. Vol. 15 (1995), pp. 547-551; Relton, J. K., Brain Res. Bull. Vol. 29 (1992), pp. 243-246; Yamasaki, Y. et al. Stroke, Vol. 26 (1995), pp. 676-680). Furthermore, mice with decreased IL-1β production (caspase-1 knockouts) are significantly protected from ischemic injury (Schielke, G. P. et al. J. Cereb. Blood Flow Metab. Vol. 18 (1998), pp. 180-185) and IL-1^{α} and β double knockouts exhibit dramatically reduced ischemic infarct volumes compared with wild-type mice (87% reduction in cortex) (Boutin, H. et al. J. Neurosci. Vol. 21 (2001), pp. 5528-5534).

In addition to a role in ischemic damage, IL-1 elevation has been associated with many neurodegenerative diseases. There is increasing evidence for a role of IL-1 in Alzheimer's disease (AD) (Mrak, R. E. et al. Neurobiol. Aging, Vol. 22, no. 6 (2001), pp. 903-908). Elevated levels of IL-1β have been shown to surround amyloid plaques in the disease and recent genetic studies have indicated that a polymorphism in IL-1^{α} is linked to an increased risk of AD (3-6 fold increase) (Griffin, W. S. et al. J. Leukoc. Biol. Vol. 72, no. 2 (2002), pp. 233-238). This polymorphism has also been correlated with rate of cognitive decline in AD patients (Murphy, G. M. et al. Neurology, Vol. 56, no. 11 (2001), pp. 1595-1597). The risk of AD is increased even further when the polymorphism in IL-1.alpha. is found in combination with another polymorphism in IL-1β (see Griffin, W. S., supra), providing convincing evidence that these cytokines play an important role in the pathology of the disease.

This assay measures the release of IL-1β from a mouse microglial cell line following an inflammatory challenge with LPS and interferon-gamma. The ability of test articles to inhibit microglial cell activation and IL-1β release is determined by co-incubation of the test article with the inflammatory challenge.

Cerebral ischemic insults are modeled in animals by occluding vessels to, or within, the cranium (Molinari, G. F. in: Barnett, H. J. M. et al. (Eds.), Stroke: Pathophysiology, Diagnosis and Management, Vol. 1 (New York, Churchill Livingstone, 1986). The rat middle cerebral artery occlusion (MCAO) model is one of the most widely used techniques to induce transient focal cerebral ischemia approximating cerebral ischemic damage in humans, e.g., those who suffer from a stroke. The middle cerebral artery used as the ischemic trigger in this model is the most affected vessel in human stroke. The model also entails a period of reperfusion, which typically occurs in human stroke victims. MCAO involving a two-hour occlusion has been found to produce the maximum size of cortical infarction obtainable without increased mortality at twenty-four hours.

Administration

The compounds of the invention are administered at a therapeutically effective dosage, e.g., a dosage sufficient to provide treatment for the disease states previously described. Administration of the compounds of the invention or the pharmaceutically acceptable salts thereof can be via any of the accepted modes of administration for agents that serve similar utilities.

While human dosage levels have yet to be optimized for the compounds of the invention, a dose may be from about 1 mg to 1 g, preferably 10 mg to 500 mg and most preferably 10 mg to 100 mg per administration. The amount of active compound administered will, of course, be dependent on the subject and disease state being treated, the severity of the affliction, the manner and schedule of administration, and the judgment of the prescribing physician.

In employing the compounds of this invention for treatment of the above conditions, any pharmaceutically acceptable mode of administration can be used. The compounds of this invention can be administered either alone or in combination with other pharmaceutically acceptable excipients, including solid, semi-solid, liquid or aerosol dosage forms, such as, for example, tablets, capsules, powders, liquids, suspensions, suppositories, aerosols or the like. The compounds of this invention can also be administered in sustained or controlled release dosage forms, including depot injections, osmotic pumps, pills, transdermal (including electrotransport) patches, and the like, for the prolonged administration of the compound at a predetermined rate, for example, in unit dosage forms suitable for single administration of precise dosages. The compositions will typically include a conventional pharmaceutical carrier or excipient and a compound of this invention or a pharmaceutically acceptable salt thereof. In addition, these compositions may include other medicinal agents, pharmaceutical agents, carriers, adjuvants, and the like, including, but not limited to, anticoagulants, blood clot dissolvers, permeability enhancers, and slow release formulations.

Generally, depending on the intended mode of administration, the pharmaceutically acceptable composition will contain about 0.1% to 90%, for example about 0.5% to 50%, by weight of a compound or salt of this invention, the remainder being suitable pharmaceutical excipients, carriers, etc.

One manner of administration for the conditions detailed above is oral, using a convenient daily dosage regimen which can be adjusted according to the degree of affliction. For such oral administration, a pharmaceutically acceptable, non-toxic composition is formed by the incorporation of any of the normally employed excipients, such as, for example, mannitol, lactose, starch, magnesium stearate, sodium saccharine, talcum, cellulose, sodium crosscarmellose, glucose, gelatin, sucrose, magnesium carbonate, and the like. Such compositions take the form of solutions, suspensions, tablets, dispersible tablets, pills, capsules, powders, sustained release formulations, and the like.

Certain compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose and derivatives thereof, and the like.

Liquid pharmaceutically administrable compositions can, for example, be prepared by dissolving, dispersing, etc. an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, sodium acetate, sodium citrate, cyclodextrine derivatives, sorbitan monolaurate, triethanolamine acetate, triethanolamine oleate, etc. Actual methods of preparing such dosage forms are known, or will be apparent, to those skilled in this art; for example, see Remington's Pharmaceutical Sciences, 15^{th} Edition, Easton, Pa., Mack Publishing Company, 1975. The composition or formulation to be administered will, in any event, contain a quantity of the active compound in an amount effective to alleviate the symptoms of the subject being treated. Dosage forms or compositions containing active ingredient in the range of 0.005% to 95% with the balance made up from non-toxic carrier may be prepared.

For a solid dosage form, the solution or suspension in for example, propylene carbonate, vegetable oils or triglycerides, is encapsulated in a gelatin capsule. Such diester solutions, and the preparation and encapsulation thereof, are disclosed in U.S. Pat. Nos. 4,328,245; 4,409,239; and 4,410,545. For a liquid dosage form, the solution, e.g. in a polyethylene glycol, may be diluted with a sufficient quantity of a pharmaceutically acceptable liquid carrier, e.g. water, to be easily measured for administration.

Alternatively, liquid or semi-solid oral formulations may be prepared by dissolving or dispersing the active compound or salt in vegetable oils, glycols, triglycerides, propylene glycol esters (e.g. propylene carbonate) and the like, and encapsulating these solutions or suspensions in hard or soft gelatin capsule shells.

The formulation can be administered in a single unit dosage form for continuous treatment or in a single unit dosage form ad libitum when relief of symptoms is specifically required. For example, the formulation may be administered as a bolus or as a continuous intravenous infusion after onset of symptoms of stroke, myocardial infarction or chronic heart failure.

Another manner of administration is the topical administration. “Topical administration” refers to application of the present compositions by spreading, spraying, etc. onto the surface of the skin. The typical amount applied may vary from about 0.1 mg of composition per square centimeter of skin to about 25 mg of composition per square centimeter of skin. Certain compounds of the present invention may be formulated for topical administration to the epidermis as ointments, creams or lotions, or as transdermal patch. Formulations suitable for topical administration in the mouth include lozenges, pastilles and mouthwashes.

The topical formulations of the present invention can be formulated as solutions. Solutions typically include an aqueous solvent (e.g., from about 50% to about 99.99% or from about 90% to about 99% of a cosmetically acceptable aqueous solvent).

As used herein, “cosmetically acceptable carrier” or “pharmaceutically acceptable carrier” includes any and all solvents, dispersion media, coatings, antibacterial and antifungal agents, isotonic and absorption delaying agents and the like. The use of such media and agents for dermatologically active substances is well known in the art. Except insofar as any conventional media or agent is incompatible with the active ingredient, its use in the therapeutic compositions is contemplated. Supplementary active ingredients can also be incorporated into the compositions.

For topical administration, the subject compositions may be provided as a wide variety of product types including, but not limited to, lotions, creams, serums, gels, sticks, sprays, mousses, foams, emollients, ointments, pastes, shampoos and conditioners. These product types may comprise several types of formulations including, but not limited to, solutions, emulsions, gels, solids, and liposomes.

Compositions useful for topical administration of the compositions of the present invention formulated as solutions typically include a cosmetically or pharmaceutically acceptable aqueous or organic solvent. Solutions typically include an aqueous solvent (e.g., from about 50% to about 99.99% or from about 90% to about 99% of a cosmetically acceptable aqueous solvent). A lotion can be made from such a solution. Lotions typically comprise from about 1% to about 20% (e.g., from about 5% to about 10%) of an emollient(s) and from about 50% to about 90% (e.g., from about 60% to about 80%) of water. Another type of product that may be formulated from a solution is a cream. A cream typically comprises from about 5% to about 50% (e.g., from about 10% to about 20%) of an emollient(s) and from about 45% to about 85% (e.g., from about 50% to about 75%) of water. An ointment may comprise from about 2% to about 10% of an emollient(s) plus from about 0.1% to about 2% of a thickening agent(s). A more complete disclosure of thickening agents or viscosity increasing agents useful herein can be found in the ICI Handbook pp. 1693-7.

The topical formulations of this invention can also be formulated as a gel (e.g., an aqueous gel using a suitable gelling agent). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprise between about 0.1% and 5%, by weight, of such gelling agents.

If the topical composition useful in the subject invention is formulated as an aerosol and applied to the skin as a spray-on, a propellant may be added to a solution composition. Examples of propellants useful herein include, but are not limited to, the chlorinated, fluorinated, and chloro-fluorinated lower molecular weight hydrocarbons, such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin.

Topical compositions useful in the subject invention may be formulated as a solution comprising an emollient. As used herein, “emollients” refer to materials used for the prevention or relief of dryness, as well as for the protection of the skin. A wide variety of suitable emollients is known and may be used herein. Representative emollients useful in the present invention include, but are not limited to, emollients that are petroleum-based; sucrose ester fatty acids; polyethylene glycol and derivatives thereof; humectants; fatty acid ester type; alkyl ethoxylate type; fatty acid ester ethoxylates; fatty alcohol type; polysiloxane type; propylene glycol and derivatives thereof; glycerine and derivatives thereof, including glycerides, acetoglycerides, and ethoxylated glycerides of C₁₂-C₂₈ fatty acids; triethylene glycol and derivatives thereof; spermaceti or other waxes; fatty acids; fatty alcohol ethers, particularly those having from 12 to 28 carbon atoms in their fatty chain, such as stearic acid; propoxylated fatty alcohols; other fatty esters of polyhydroxy alcohols; lanolin and its derivatives; kaolin and its derivatives; any of the monographed skin care agents listed above; or mixtures of these emollients. Suitable petroleum-based emollients include those hydrocarbons, or mixtures of hydrocarbons, having chain lengths of from 16 to 32 carbon atoms. Petroleum based hydrocarbons having these chain lengths include mineral oil (also known as “liquid petrolatum”) and petrolatum (also known as “mineral wax,” “petroleum jelly,” and “mineral jelly”). Mineral oil usually refers to less viscous mixtures of hydrocarbons having from 16 to 20 carbon atoms. Petrolatum usually refers to more viscous mixtures of hydrocarbons having from 16 to 32 carbon atoms. Petrolatum and mineral oil are particularly preferred emollients for compositions of the present invention. Formulations for these types of products are well known in the art and some are described for example in The International Cosmetic Ingredient Dictionary and Handbook, eds. Wenninger and McEwen, pp. 1656-61, 1626, and 1654-5 (“ICI Handbook”) which contains numerous examples of suitable materials. Such compositions preferably contain from about 2% to about 50% of an emollient.

The topical formulations may comprise one or more cosmetic agents as defined herein. The cosmetic agent will typically be present in the formulation of the invention in an amount of from about 0.001% to about 20% by weight of the formulation, e.g., about 0.01% to about 10% such as about 0.1% to about 5%.

Yet another type of product that may be formulated from a composition of the present invention is an ointment. An ointment may comprise a simple base of animal or vegetable oils or semi-solid hydrocarbons (oleaginous). Ointments may also comprise absorption ointment bases which absorb water to form emulsions. Ointment carriers may also be water soluble.

Another type of formulation is an emulsion. Emulsifiers may be nonionic, anionic or cationic and examples of emulsifiers are described in, for example, U.S. Pat. Nos. 3,755,560, and 4,421,769, incorporated herein by reference. Lotions and creams can be formulated as emulsions as well as solutions. Single emulsions for topical preparations, such as lotions and creams, of the oil-in-water type and water-in-oil type are well-known in the art. Multiphase emulsion compositions, such as the water-in-oil-in-water type, are also known, as disclosed, for example, in U.S. Pat. No. 4,254,105. Triple emulsions are also useful for topical administration of the present invention and comprise an oil-in-water-in-silicone fluid emulsion as disclosed, for example in U.S. Pat. No. 4,960,764.

Another emulsion useful in the topical compositions is a micro-emulsion system. For example, such a system comprises from about 9% to about 15% squalane, from about 25% to about 40% silicone oil, from about 8% to about 20% of a fatty alcohol, from about 15% to about 30% of polyoxyethylene sorbitan mono-fatty acid (commercially available under the trade name TWEENS) or other nonionics, and from about 7% to about 20% water.

Liposomal formulations are also useful for the compositions of the present invention. Such compositions can be prepared by combining a composition of the present invention with a phospholipid, such as dipalmitoylphosphatidyl choline, cholesterol and water according to known methods, for example, as described in Mezei et al. J. Pharm. Pharmacol., Vol. 34 (1982), pp. 473-4, or a modification thereof. Lipids suitable for forming liposomes may be substituted for the phospholipid, as may be lecithin, as well. The liposome preparation is then incorporated into one of the above topical formulations (for example, a gel or an oil-in-water emulsion) in order to produce the liposomal formulation. Other compositions and pharmaceutical uses of topically applied liposomes are described for, example, in Mezei. Topics in Pharmaceutical Sciences (New York, Breimer et al. eds., Elsevier Science, 1985), pp. 345-58.

This invention also includes compositions described above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the oral compositions discussed above can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g., about 40 mesh.

Some examples of suitable excipients for oral preparations include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the subject by employing procedures known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound may be effective over a wide dosage range and is generally administered in a pharmaceutically or cosmetically effective amount, as described above. It, will be understood, however, that the amount of the compound actually administered will, in the case of a pharmaceutical, be determined by a physician; in the light of the relevant circumstances; including the condition to be treated; the chosen route of administration; the actual compound administered; the age, weight, and response of the individual subject; the severity of the subject's symptoms; and the like. In the case of a cosmetic or over-the-counter skin care preparation, the actual amount of compound desired to be administered by the consumer will be recommended by the manufacturer; based on the manufacturer's test results, which may, in whole or in part, be determined on the basis of one or more of the in vitro and/or in vivo tests described herein.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents, solubility enhancers, and the like, such as, for example, sodium acetate, sorbitan monolaurate, triethanolamine oleate, cyclodextrins, etc.

Another approach for parenteral administration employs the implantation of a slow-release or sustained-release system, such that a constant level of dosage is maintained. The percentage of active compound contained in such parenteral compositions is highly dependent on the specific nature thereof, as well as the activity of the compound and the needs of the subject. However, percentages of active ingredient of 0.01% to 10% in solution are employable, and will be higher if the composition is a solid which will be subsequently diluted to the above percentages.

Nasal solutions of the active compound alone or in combination with other pharmaceutically acceptable excipients can also be administered.

Formulations of the active compound or a salt may also be administered to the respiratory tract as an aerosol or solution for a nebulizer, or as a microfine powder for insufflation, alone or in combination with an inert carrier such as lactose. In such a case, the particles of the formulation have diameters of less than 50 microns, for example less than 10 microns.

### EXAMPLES

The following preparations and examples are given to enable those skilled in the art to more clearly understand and to practice the present invention. They should not be considered as limiting the scope of the invention, but merely as being illustrative and representative thereof.

General Characterization Methods

As reported in the following examples, Nuclear Magnetic Resonance (NMR) spectra were recorded on a Bruker DTX 300 spectrometer using, in most cases, tetramethyl silane (TMS) as the internal reference. Mass spectra were obtained on an Agilent 1100 LC/MSD instrument using either electrospray ionization (positive or negative mode) (ESI) or atmospheric pressure chemical ionization (positive or negative mode) (APCI).

Further, abbreviations used throughout the specification have the following meanings:
br s=broad singlet
cc=cubic centimeters, milliliters
d=doublet
dd=doublet of doublets
DMSO=dimethylsulfoxide
ELISA=enzyme-linked immunosorbant assay
Et=ethyl
EtOAc=ethyl acetate
EtOH=ethanol
FBS=fetal bovine serum
g=gram
h=hour
Hz=Hertz
I. P.=intraperitoneal
I.V.=intravenous
IC₅₀=The molar concentration of a drug, which produces 50% of the maximum possible inhibition for that drug
kg=kilogram
LPS=lipopolysaccharide
M=Molar
m=multiplet
m/z=mass-to-charge ratio
Me=methyl
MeOH=methanol
mg=milligram
MHz=mega Hertz
min=minute
mL=milliliter
mM=millimolar
mmol=millimole
N=normal
NMR=nuclear magnetic resonance
PBS=phosphate buffered saline
ppm=parts per million
psi=pounds per square inch
s=singlet
t=triplet
v/v=volume/volume
μg=microgram
μL=microliter
μM=micromolar
μmol=micromole

### Example 1

### 6-hydroxy-2,2,5,7,8-pentamethyl-4-hydroxy-chroman

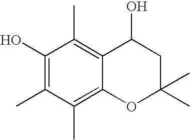

### Step 1: 2,3,5-trimethyl-1,4-phenylene bis(3-methylbut-2-enoate)

To a solution of 2,3,5-trimethylbenzene-1,4-diol (20 g) in 150 mL of toluene was added 3-methylbut-2-enoyl chloride (30 mL). The reaction mixture was allowed to reflux for 2-3 hours. The mixture was extracted with ethyl acetate, washed with NaHCO₃ and dried over anhydrous Na₂SO₄. After concentrated in vacuo, crystallization of the resulting residue from ethyl acetate and hexane gave 32 g of 2,3,5-trimethyl-1,4-phenylene bis(3-methylbut-2-enoate) as a white solid.

### Step 2: 6-hydroxy-2,2,5,7,8-pentamethylchroman-4-one

The above ester (30 g) and anhydrous AlCl₃ (13.9 g) were mixed and heated to 140° C. for 2 hours. During this time, the mixture turned dark-brown melt. After allowing it to cool, the melt was dissolved in 300 mL of dichloromethane. To the solution was added slowly 100 mL of 1N HCl. The organic phase was separated, and washed with NaHCO₃ and dried over anhydrous Na₂SO₄. After concentration in vacuo, the dark brown residue (37 g) was suspended in 150 mL of 1N NaOH in MeOH/water and was refluxed for 2 hours. The solution was cooled down, acidified with 1N HCl, and then extracted with ethyl acetate. The organic layer was washed with NaHCO₃, dried over anhydrous Na₂SO₄, and concentrated in vacuo. Crystallization of the resulting residue from ethyl acetate and hexane gave 17.9 g of 6-hydroxy-2,2,5,7,8-pentamethylchroman-4-one as a yellow solid.

### Step 3: 6-hydroxy-2,2,5,7,8-pentamethyl-4-hydroxy-chroman

To a solution of 6-hydroxy-2,2,5,7,8-pentamethyl-chroman-4-one (156 mg) in 5 mL of MeOH was added sodium borohydride (51 mg). The reaction was allowed to stir for 1 hour. After the reaction was acidified with 1N HCl, the mixture was concentrated and with ethyl acetate. The organic layer was washed with water and dried over anhydrous Na₂SO₄. After concentration in vacuo, the resulting residue was purified by flash chromatography eluted with 30% ethyl acetate in hexane to give 125 mg of 6-hydroxy-2,2,5,7,8-pentamethyl-4-hydroxy-chroman as a light-yellow solid.

¹H NMR (300 MHz, CD₃OD) 4.85 (t, 1H), 4.64 (s, 1H), 2.26 (s, 3H), 2.15 (s, 3H), 2.07 (s, 3H), 2.01 (d, 2H), 1.37 (s, 3H), 1.33 (s, 3H). ¹³C NMR (75 MHz, CD₃OD) 145.4, 145.3, 125.8, 122.4, 118.6, 72.6, 62.0, 42.7, 28.5, 26.0, 12.2, 11.6, 11.5. MS: m/z=219.1 (M+H⁺−18), 259.1 (M+Na⁺).

### 2,2,7,8-tetramethylchroman-4,6-diol

Similarly to a solution of 6-hydroxy-2,2,7,8-tetramethylchroman-4-one (50 mg) in MeOH (10 mL) was added sodium borohydride (40 mg). The solution was stirred at room temperature overnight, then poured into water and extracted with EtOAc. The EtOAc was washed with water and dried over MgSO₄, and evaporated. The residue was purified by eluting on a silica gel column with 50% EtOAc in hexane to give 25 mg of 2,2,7,8-tetramethylchroman-4,6-diol: ¹H NMR (300 MHz, CDCl₃) δ=6.76 (s, 1H), 5.29 (br s, 1H), 4.75 (m, 1H), 2.16, 2.09 (2s, 6H), 1.78 (m, 2H), 1.41, 1.25 (2s, 6H) ppm. ¹³C NMR (CDCl₃, 75 MHz) δ=147.50, 144.38, 125.54, 124.39, 121.15, 109.85, 74.43, 63.68, 49.34, 48.74, 42.52, 29.06, 25.47, 11.94, 11.90 ppm. MS (m/z)=205 (M+H⁺).

### Example 2

### 4-Methoxyamino-2,2,5,7,8-pentamethyl-chroman-6-ol

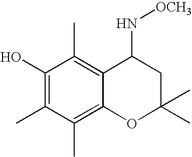

### Step 1: 6-Hydroxy-2,2,5,7,8-pentamethyl-chroman-4-one O-methyl-oxime

A mixture of 6-hydroxy-2,2,5,7,8-pentamethyl-chroman-4-one (234 mg) prepared as described in Example 3 for the thiochroman analog, but substituting 4-mercapto-2,3,6-trimethyl-phenol with 2,3,5-trimethyl-benzene-1,4-diol, and MeONH₂. HCl (250 mg) in 8 mL of pyridine was vigorously stirred for 15 h and concentrated. The residue was washed with water and chromatographed to afford 6-hydroxy-2,2,5,7,8-pentamethyl-chroman-4-one O-methyl-oxime as a brown oil (250 mg).

¹H-NMR (300 Hz, CDCl₃) δ=4.59 (s, 1H), 4.02 (s, 3H), 2.86 (s, 2H), 2.54 (s, 3H), 2.22 (s, 3H), 2.15 (s, 3H), 1.37 (s, 6H) ppm. ¹³C NMR (75 Hz, CDCl₃) δ=151.9, 147.6, 146.0, 125.9, 123.6, 118.6, 114.7, 74.0, 61.9, 35.8, 27.0, 14.8, 12.8, 12.0 ppm. (ESI) m/z: 264 (M+H⁺).

### Step 2: 4-methoxyamino-2,2,5,7,8-pentamethyl-chroman-6-ol

To a solution of 6-hydroxy-2,2,5,7,8-pentamethyl-chroman-4-one O-methyl-oxime (131 mg) in 5 mL EtOH was added BH₃.pyridine complex (139 mg) at O ° C. followed by addition of concentrated HCl (0.16 mL). The reaction was stirred at room temperature for 15 h and quenched on ice. It was neutralized with NaHCO₃ (concentrated) and extracted with EtOAc (3×30 mL). The organic layers were dried over Na₂SO₄ and concentrated and the crude product was chromatographed to afford 4-methoxyamino-2,2,5,7,8-pentamethyl-chroman-6-ol as a brown wax (92 mg).

¹H-NMR (300 Hz, CDCl₃) δ=4.54 (s, 1H), 4.32 (m, 1H), 3.63 (s, 3H), 2.35-2.30 (m, 4H), 2.14 (s, 3H), 2.09 (s, 3H), 1.95 (dd, J=14.2, 5.9 Hz, 1H), 1.55 (s, 3H), 1.34 (s, 3H) ppm; ¹³C NMR (75 Hz, CDCl₃) δ=146.5, 145.5, 123.67, 123.61, 119.4, 116.0, 73.6, 62.0, 52.9, 37.6, 29.2, 28.2, 12.4, 11.9, 11.7 ppm; (ESI) m/z: 219 (M−MeONH⁻).

### Example 3

### 6-Hydroxy-2,2,5,7,8-pentamethyl-thiochroman-4-one O-methyl-oxime

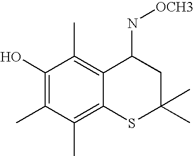

### Step 1: 6-Hydroxy-2,2,5,7,8-pentamethyl-thiochroman-4-one

4-Mercapto-2,3,6-trimethyl-phenol (2.0 g) was dissolved in anhydrous methanol (100 mL) containing trimethyl orthoformate (2 mL), and the solution was deoxygenated by bubbling with nitrogen. To this solution was added ethyl 3,3-dimethylacrylate (8 mL) and then 5 drops of concentrated sulfuric acid. The solution was allowed to reflux for 6 days. The mixture was concentrated, washed with NaHCO₃ and extracted with ethyl acetate. After concentrated in vacuo, the residue was purified by flash chromatography eluted with 20% ethyl acetate in hexane to give 906 mg of 3-(4-hydroxy-2,3,5-trimethyl-phenylsulfanyl)-3-methyl-butyric acid methyl ester as a white solid. The ester was suspended in 100 mL of 1N NaOH in MeOH and water (1:1, v/v), and the mixture was stirred for 1 hour. The mixture was acidified with 1N HCl and extracted 3 times with ethyl acetate. The organic layer was washed with water, dried over anhydrous MgSO₄, and concentrated in vacuo to give the correspondent acid, 3-(4-hydroxy-2,3,5-trimethyl-phenylsulfanyl)-3-methyl-butyric acid, which was dissolved in 20 mL of concentrated sulfuric acid to form a homogeneous dark red solution. After 30 min at room temperature the solution was poured onto crushed ice. The resulting green mixture was extracted 3 times with ethyl acetate. The organic layer was washed with water and dried over anhydrous MgSO₄, and concentrated in vacuo. The residue was purified by flash chromatography eluted with 10% ethyl acetate in hexane to give 394 mg of 6-hydroxy-2,2,5,7,8-pentamethyl-thiochroman-4-one as a yellow solid. ¹H-NMR (300 Hz, CDCl₃) δ=4.84 (s, 1H), 2.86 (s, 2H), 2.50 (s, 3H), 2.27 (s, 3H), 2.26 (s, 3H), 1.46 (s, 6H) ppm.

¹³C-NMR (75 Hz, CDCl₃) δ=198.56, 149.73, 132.46, 131.75, 128.94, 128.11, 123.02, 55.48, 42.76, 29.12, 16.58, 13.83, 13.36 ppm. MS (m/z)=251.1 (M+H⁺), 273.1 (M+Na⁺).

### Step 2: 6-Hydroxy-2,2,5,7,8-pentamethyl-thiochroman-4-one O-methyl-oxime

To a solution of 6-hydroxy-2,2,5,7,8-pentamethyl-thiochroman-4-one (30 mg, 0.12 mmol) prepared as described above for in 0.5 mL of pyridine was added methoxyamine hydrochloride (15 mg, 0.18 mmol). The reaction mixture was allowed to stir overnight. The mixture was washed with water and extracted with ethyl acetate. After concentrated in vacuo, the residue was purified by flash chromatography eluted with 20% ethyl acetate in hexane to give 11 mg of 6-hydroxy-2,2,5,7,8-pentamethyl-thiochroman-4-one O-methyl-oxime as a white solid. ¹H-NMR (300 Hz, CDCl₃) δ=4.71 (s, 1H), 3.98 (s, 2H), 2.95 (s, 2H), 2.43 (s, 3H), 2.24 (s, 3H), 2.21 (s, 3H), 1.39 (s, 6H) ppm.

¹³C-NMR (75 Hz CDCl₃) δ=154.86, 150.53, 133.02, 128.18, 127.36, 123.74, 119.67, 61.98, 42.76, 42.27, 29.87, 16.69, 14.46, 12.81 ppm. MS (m/z)=280.1 (M+H⁺).

### Example 4

### 6-hydroxy-2,2,5,7,8-pentamethylchroman-3-one O-methyl oxime

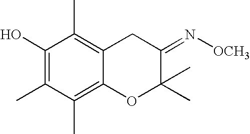

To 2.2 g of 2,2,5,7,8-pentamethylchroman-6-ol (10 mmol) in 50 mL dichloromethane was added triethylamine (30 mmol) and then acetyl chloride (20 mmol), dropwise. The reaction was stirred at room temperature for 1 h and concentrated. The residue was diluted with EtOAc (80 mL) and washed with water (3×50 mL) and HCl (0.5 M, 3×50 mL) to afford 2,2,5,7,8-pentamethylchroman-6-yl acetate. MS (m/z)=263 (100, M+H⁺).

A toluene solution of 2,2,5,7,8-pentamethylchroman-6-yl acetate was heated to reflux for 30 min followed by a slow addition of a solution of 2,3,dicloro-5,6-dicyano-1,4-benzoquinone (20 mmol) in toluene slowly. The reaction was refluxed for 15 h and concentrated. The crude material was chromatographed to afford the desired 2,2,5,7,8-pentamethyl-2H-chromen-6-yl acetate (2.2 g). MS (m/z)=261 (100, M+H⁺).

To a solution of 2,2,5,7,8-pentamethyl-2H-chromen-6-yl acetate (1.3 g, 5 mmol) in 25 mL methanol was added a 10% NaOH solution (4 mL, 10 mmol). The mixture was stirred vigorously for 1 h and neutralized with concentrated NaH₂PO₄ solution. It was extracted with EtOAc (3×30 mL) and the combined organic phase was dried over Na₂SO₄ and concentrated to afford 2,2,5,7,8-pentamethyl-2H-chromen-6-ol. MS (m/z)=219 (100, M+H⁺).

To a solution of 2,2,5,7,8-pentamethyl-2H-chromen-6-ol (300 mg, 1.37 mmol) and imidazole (186 mg, 2.74 mmol) in 5 mL dichloromethane and 2 mL dimethylformamide was added t-butyldimethylsilyl chloride (411 mg, 2.74 mmol). The resulting mixture was stirred for 15 hours and concentrated. The crude product was purified by chromatography (415 mg).

To above protected chroman (100 mg, 0.3 mmol) in 5 mL dichloromethane at 0° C. was added m-chloroperoxybenzoic acid (CPBA) (89 mg, 0.36 mmol). The reaction was stirred at room temperature for 3 hours and quenched by adding 30 mL ice. It was extracted with ethyl acetate (3×20 L) and the organic phase was dried over Na₂SO₄ and concentrated. The crude product was purified by chromatography to yield 6-(tert-butyldimethylsilyloxy)-3-hydroxy-2,2,5,7,8-pentamethylchroman-4-yl 3-chlorobenzoate (102 mg).

To this ester (100 mg, 0.2 mmol) in 5 mL dry tetrahydrofuran was added AlCl₃ (840 mg, 0.6 mmol) and LiAlH₄ (0.8 mL, 0.4 mmol) at room temperature. The reaction was stirred for 2 h and quenched by adding ice (30 g). It was extracted with EtOAc (3×20 mL) and the organic phase was dried over Na₂SO₄ and concentrated. The crude product was purified by chromatography to afford two diastereoisomers of 6-(tert-butyldimethylsilyloxy)-2,2,5,7,8-pentamethylchroman-3,4-diol (cis 23 mg, trans 36 mg).

The cis isomer (23 mg, 0.06 mmol) in 5 mL MeOH in the presence of Pd/C was hydrogenated at 55 psi for 15 h and concentrated to give 6-(tert-butyldimethylsilyloxy)-2,2,5,7,8-pentamethylchroman-3-ol. To this crude material in 2 mL of dichloromethane was added Dess-Martin periodinane (0.12 mmol) at 0° C. and the reaction was allowed to warm to room temperature and stirring was continued for 1 hour. The reaction mixture was concentrated and the residue was filtered through a short silica gel column to afford 6-(tert-butyidimethylsilyloxy)-2,2,5,7,8-pentamethylchroman-3-one (13 mg).

To 6-(tert-butyldimethylsilyloxy)-2,2,5,7,8-pentamethylchroman-3-one in 2 mL of tetrahydrofuran was added tetrabutylammonium fluoride (1 mmol) at 0° C. and the reaction was allowed to warm to room temperature, stirred for 2 h and concentrated. The product was purified by filtering through a short silica gel column to afford the desired 6-hydroxy-2,2,5,7,8-pentamethylchroman-3-one (6 mg). MS (m/z)=235 (100, M+H⁺).

A mixture of 6-hydroxy-2,2,5,7,8-pentamethylchroman-3-one and methoxyamine (12 mg) in 2 mL EtOH and 1 mL pyridine was heated to reflux for 2 h and concentrated and dried under high vacuum. The crude product was purified by chromatography to afford 6-hydroxy-2,2,5,7,8-pentamethylchroman-3-one O-methyl oxime (4.5 mg). ¹H-NMR (300 MHz, CDCl₃) δ=4.33 (s, 1H), 3.93 (s, 3H), 3.57 (s, 2H), 2.19 (s, 3H), 2.17 (s, 3H), 1.60 (s, 3H), 1.46 (s, 6H) ppm. ¹³C NMR (75 MHz, CDCl₃) δ=158.5, 145.9, 144.4, 123.7, 121.1, 118.3, 117.5, 75.4, 61.7, 25.4, 23.0, 11.9, 11.4 ppm. MS (m/z)=264 (M+H⁺).

### Example 5

### 5-Lipoxygenase Enzyme Assay

This procedure was used for measuring the enzymatic activity of human recombinant 5-lipoxygenase using a colorimetric method based on the ferric oxidation of xylenol orange.

Materials

96 well flat bottom microfilter plates (VWR, Catalog # 62402-933 9295)

Lipoxygenase screening assay buffer (Cayman, Catalog # 760710)

Human recombinant 5-lipoxygenase (Cayman, Catalog # 60402)

Arachidonic Acid (Sigma, Catalog # A3555)

Xylenol orange tetrasodium salt (Aldrich, Catalog # 227854)

Iron (II) sulfate heptahydrate (Sigma, Catalog # F7002)

Sulfuric acid (95-98%) [18M]

Methanol

Procedure

Human recombinant 5-lipoxygenase (Cayman Cat # 60402) was used in this assay. The test compound and/or vehicle was added to 0.5 μL 5-lipoxygenase in 50 mM Tris-HCl buffer, pH 7.4. The reaction was initiated by addition of 70 μM arachidonic acid in Tris-HCl buffer, pH 7.4, and terminated after a 10 minute incubation at room temperature by addition of FOX reagent (25 mM sulfuric acid, 100 μM xylenol orange, 100 μM iron (II) sulphate, methanol:water 9:1). The yellow color of acidified xylenol orange was converted to a blue color by the lipid hydroperoxide-mediated oxidation of Fe²⁺ ions and the interaction of the resulting Fe³⁺ ions with the dye. The complex was allowed to form during a 1 hour incubation at room temperature with shaking. Absorbance of the Fe³⁺ complex was then measured at 620 nM using a spectrophotometer.

Negative controls contained enzyme during the incubation step but substrate was not added until after the FOX reagent. Compounds were screened at 5 concentrations in triplicate starting at 10 μM.

Certain compounds of the present invention such as:
6-Hydroxy-2,2,5,7,8-pentamethyl-chroman-4-one O-methyl-oxime;
6-Hydroxy-2,2,5,7,8-pentamethyl-thiochroman-4-one O-methyl-oxime;
4-Methoxyamino-2,2,5,7,8-pentamethyl-chroman-6-ol;
6-Hydroxy-2,2,5,7,8-pentamethyl-2,3-dihydro-4H-chromen-4-one dimethylhydrazone;
2,2,5,7,8-pentamethylchroman-4,6-diol; and
2,2,7,8-tetramethylchroman-4,6-diol
   were considered to be active when they exhibited inhibition of 5-Lipoxygenase with an IC₅₀ in a range of less than about 3 μM.

### Example 6

### 12/15-Lipoxygenase Enzyme Assay

This procedure was used for measuring the enzymatic activity of porcine leukocyte 12/15-lipoxygenase using a colorimetric method based on the ferric oxidation of xylenol orange.

Materials

96 well flat bottom microfilter plates (VWR, Catalog # 62402-933 9295)

Lipoxygenase screening assay buffer (Cayman, Catalog #760710)

Porcine leukocyte 12/15-lipoxygenase (Cayman, Catalog #60300)

Arachidonic Acid (Sigma, Catalog # A3555)

Xylenol orange tetrasodium salt (Aldrich, Catalog # 227854)

Iron (II) sulfate heptahydrate (Sigma, Catalog # F7002)

Sulfuric acid (95-98%) [18M]

Methanol

Procedure

Porcine Leukocyte 12/15-lipoxygenase (Cayman Cat # 60300) was used in this assay. Test compound and/or vehicle were added to 1.3 μL 12/15-lipoxygenase in 50 mM Tris-HCl buffer, pH 7.4. The reaction was initiated by addition of 70 μM arachidonic acid in Tris-HCl buffer, pH 7.4 and terminated after a 10 minute incubation at room temperature by addition of FOX reagent (25 mM sulfuric acid, 100 μM xylenol orange, 100 μM iron (II) sulphate, methanol:water 9:1). The yellow color of acidified xylenol orange was converted to a blue color by the lipid hydroperoxide-mediated oxidation of Fe²⁺ ions and the interaction of the resulting Fe³⁺ ions with the dye. The complex was allowed to form during a 1 hour incubation at room temperature with shaking. Absorbance of the Fe³⁺ complex was then measured at 620 nM using a spectrophotometer.

Negative controls contained enzyme during the incubation step but substrate was not added until after the FOX reagent.

Compounds are screened at 5 concentrations in triplicate starting at 10 μM.

Certain compounds of the present invention such as:
6-Hydroxy-2,2,5,7,8-pentamethyl-chroman-4-one O-methyl-oxime;
6-Hydroxy-2,2,5,7,8-pentamethyl-thiochroman-4-one O-methyl-oxime;
4-Methoxyamino-2,2,5,7,8-pentamethyl-chroman-6-ol;
6-Hydroxy-2,2,5,7,8-pentamethylchroman-3-one O-methyl oxime
2,2,5,7,8-pentamethylchroman-4,6-diol
   exhibited inhibition of 12/15-Lipoxygenase with an IC₅₀ in a range of less than 5 μM.

### Example 7

### Inhibition of LTB₄ Production in Blood

The following materials were used in this protocol.

Materials

Human whole blood (Na citrate) (Stanford Blood Center)

A23187, (Sigma, Cat # C-7522)

Leukotriene B4 EIA reagents (Cayman Chemical, Cat # 520111)

BWA4C (Sigma, Cat # B7559)

Procedure

Preparation of A23187:

A23187 was prepared as a 10 mM stock solution in DMSO (aliquots can be stored at −20° C.). On the day of the assay the stock solution was diluted as follows: 70 μL 10 mM stock added to 1.6 mL plasma to give a working concentration of 0.42 mM.

Preparation of Test Articles:

From a 30 mM stock solution in DMSO, test articles were diluted to a working concentration of 600 μM in PBS (i.e. 10 μL stock solution+490 μL PBS). This is the highest concentration (gives a final testing concentration of 30 μM). From this 600 μM solution test articles were serially diluted 1:3 in PBS to give a dose-response curve. 10 μL of each concentration of test article was then added to 4 wells of a 96-well plate (i.e. testing in quadruplicate). A positive control compound, BWA4C was used in every assay.

Blood Stimulation Procedure

Human whole blood was added to the plates containing compounds (190 μL per well) and mixed well. The blood was incubated with compound at 37° C. for 15 minutes. Following this incubation, 10 μL of 0.42 mM A23187 was added to each well except the negative control wells to give a final calcium ionophore concentration of 20 μM. The plates were then incubated at 37° C. for 60 minutes. After the incubation period, plates were centrifuged for 15 minutes at 2000 g at 4° C. in sealed microplate buckets. Plasma was then removed for quantitation of LTB₄ levels by ELISA.

Measurement of LTB4 Levels by ELISA

LTB₄ levels in the plasma were determined using a commercially available ELISA kit from Cayman Chemicals. The ELISA was run according to the manufacturer's instructions. The LTB₄ levels in the vehicle control sample were then compared to those in which the test article had been added. From this a percent inhibition of LTB₄ production by each concentration of test article was calculated and the IC₅₀ was determined.

Certain compounds of this invention when tested as described provided protection against LTB₄ at an IC₅₀ of less than 5 μM.

### Example 8

### LTB₄-Cell Assay

This procedure was used for measuring the release of the leukotriene LTB₄ from a neutrophil cell line using a competitive ELISA technique.

Materials and Equipments

Materials for Cell Preparation and Experiment

MPRO cell line (ATCC, Catalog # CRL-11422)

Calcium ionophore (A23187) (Sigma, Catalog # C7522)

Nordihydroguaiaretic acid (NDGA) (BioMol, Catalog # EI101-0001)

Retinoic Acid (all-trans) (ATRA) (Sigma, Catalog # 95152)

Sterile, tissue-culture treated 96-well plates (Corning, Catalog # 3614)

Materials for LTB4 ELISA

Precoated (Mouse Anti-Rabbit IgG) EIA 96 Well Strip Plates (Cayman, Catalog # 400004)

Leukotriene B4 AChE Tracer (Cayman Catalog # 420110)

Leukotriene B4 EIA Antiserum (Cayman Catalog # 420112)

Ellman's Reagent (Cayman Catalog # 400050)

EIA Buffer Concentrate (10×) (Cayman Catalog # 400060)

Wash Buffer Concentrate (400×) (Cayman Catalog # 400062)

Plastic plate covers (Cayman Catalog # 400012)

Procedure

A mouse promyelocytic cell line (MPRO) was used in this assay. These cells are committed immature neutrophils that can be differentiated into mature neutrophils by treatment with 10 μM all-trans retinoic acid for 72 hours.

Following 72 hours of differentiation, cells were stimulated with 1 μM of a calcium ionophore (A23187) in the presence or absence of test compound or vehicle for 1 hour at 37° C. After this time, the supernatant was removed from the cells and the LTB₄ levels were determined following manufacturer's instructions, using a Leukotriene B₄ EIA kit from Cayman (Cat # 520111). The negative controls were media samples from differentiated but unstimulated cells. The compounds were screened at 5 concentrations in quadruplicate starting at 10 μM.

Following the procedure described above certain compounds of the present invention exhibited inhibition of LTB₄ Certain compounds of this invention when tested as described provided protection at an IC₅₀ of less than 5 μM.

### Example 9

### Inflammation Assay—Cell-ELAM Assay

Endothelial-Leukocyte Adhesion Molecule (ELAM), also known as E-selectin, is expressed on the surface of endothelial cells. In this assay, lipopolysaccharide (LPS) and IL-1β are used to stimulate the expression of ELAM; test agents are tested for their abilities to reduce this expression, in accordance with studies showing that reduction of leukocyte adhesion to endothelial cell surface is associated with decreased cellular damage (e.g., Takada, M., et al. Transplantation, Vol. 64 (1997), pp. 1520-25; Steinberg, J. B., et al. J. Heart Lung Trans., Vol. 13 (1994), pp. 306-313).

Endothelial cells may be selected from any of a number of sources and cultured according to methods known in the art, including, for example, coronary artery endothelial cells, human brain microvascular endothelial cells (HBMEC; Hess, D. C., et al. Neurosci. Lett., Vol. 213, no. 1 (1996), pp. 37-40), or lung endothelial cells. Cells are conveniently cultured in 96-well plates. Cells are stimulated by adding a solution to each well containing 10 μg/mL LPS and 100 μg/mL IL-1β for 6 hours in the presence of test agent (specific concentrations and time may be adjusted depending on the cell type). Treatment buffer is removed and replaced with pre-warmed Fixing Solution® (100 μL/well) for 25 minutes at room temperature. Cells are then washed 3×, then incubated with Blocking Buffer (PBS and 2% FBS) for 25 minutes at room temperature. Blocking Buffer containing Monoclonal E-Selectin Antibody (1:750, Sigma Catalog #S-9555) is added to each well. Plates are sealed and stored at 4° C. overnight. Plates are washed 4× with 160 μL Blocking Buffer per well. Second Antibody-HRP diluted 1:5000 in Blocking Buffer is then added (100 μL/well) and plates are incubated at room temperature (protected from light) for two hours. Plates are then washed 4× with Blocking Buffer before addition of 100 μL of ABTS Substrate solution at room temperature (Zymed, Catalog #00-2024). Wells are allowed to develop for 35 minutes, before measurement at 402 nm in a Fluoroskan® Reader with shake program for 10 seconds. Positive results are recorded as a decrease in ELAM concentration in tested wells, as compared to control wells.

Certain compounds of this invention when tested as described above, may show activity in this assay.

### Example 10

### Rat Paw Edema Assay

Animal Preparation:

Male Sprague-Dawley rats weighing between 175 to 200 g are used in this study. Animals are allowed free access to water and commercial rodent diet under standard laboratory conditions. Room temperature is maintained at 20-23° C. and room illumination is on a 12/12-hour light/dark cycle. Animals are acclimatized to the laboratory environment 5 to 7 days prior to the study.

Experimental Procedure:

Each animal was treated by administration of vehicle, reference or test substance one hour prior to carrageenan injection, as follows:

I.V. Infusion Via Femoral Vein:

Anesthesia is maintained by inhalation of 3.0% isoflurane (Aerane, Front Dodge, Iowa) in oxygen throughout the entire procedure. The exterior site of the right femoral vein is shaved and sterilized prior to surgery. A 3-cm incision is made in the right groin region and the femoral vein is isolated. The femoral vein is temporarily ligated with a micro-vascular clip, and a small incision is made on the femoral vein to introduce and advance a polyethylene (PE-50) catheter (Becton. Dickinson and Co., Sparks, Md.). The catheter is secured in place with suture (silk 5/0, Carlisle Laboratories, Farmers Branch, Tex.). The other end of the catheter is attached to a syringe filled with the saline for the bolus injection. Using a hemostat, a pocket is made subcutaneously on the back of the animal so the PE catheter can be brought up to the exteriorization point between the shoulder blade for either a bolus injection or a continuous injection by an osmotic pump.

I.P. Injection:

An awake rat is held in a standard hand held position. A 23 3/4G needle is injected into the lower right quarter of the abdomen pass the peritoneum, slightly off the midline. To avoid organ injection, the plunger of the syringe is slightly pulled back. If no fluid is withdrawn, the content of the syringe is delivered into the abdominal cavity.

Gavage Feeding:

A standard rat gavage tube (Popper & Sons Inc., NY) is attached to a 3-cc hypodermic syringe. The animal is held in a vertical position. The feeding tube is placed into the mouth and then gently advanced until it reached the stomach (the approximate insertion length of the tube should be measured prior to feeding). The content of the syringe is slowly delivered and then the tube is withdrawn.

One hour post treatment each animal is anesthetized with 3.0% isoflurane (Aerane, Front Dodge, Iowa) in oxygen and administered 100 μL of 1% Carrageenan Lambda type IV (Sigma Chemical Company, St. Louis, Mo.) suspension in saline, into the intraplantar surface of the right hind paw. Paw edema is measured four hours after carrageenan injection, either by measuring the increase in paw volume using a plethysmometer or the increase in paw weight using a fine scale. Immediately prior to edema measurement, the animals are euthanized via CO₂ asphyxiation and 500 μL of blood is withdrawn by cardiac puncture for later analysis. Paw volume is determined by the extent to which water is displaced by the paw from a pre-calibrated chamber. The volume of the left hind paw (control) is subtracted from the volume of the right hind paw (carrageenan-treated) to determine the volume of carrageenan-induced edema. To measure the weight difference between paws, both hind paws are removed and weighed separately.

To minimize the variation in the model, the following steps are taken:
Carrageenan is made fresh every day prior to the study (2-3 hours before injection).
The plethysmometer is calibrated each day prior to the study.
If carrageenan injection causes significant bleeding or a hematoma on the treated foot, the animal is excluded from the study.
Each paw is marked at the tibio-tarsal joint across the ankle prior to measurements, to ensure each paw was submerged at the same level.
If reading on the volume needs to be repeated, the paw has to be dried off completely.
   Statistical Analysis

The difference of the weight or the volume between right and left paw is calculated for each animal for the analysis. Group data are presented as means +/−SEM and p<0.05 are considered significant. Inter-group comparisons are carried out by unpaired student t test (between two groups) or one-way ANOVA followed by post hoc Bonferroni's multiple comparisons.

Results

Certain compounds of the present invention may show reduction in edema when tested by this methods.

### Example 11

### Mouse Ear Inflammatory Response to Topical Arachidonic Acid

Animals:

Balb C Mice 23-28 g, from Simonsen Labs, Gilroy, Calif.

Materials:

Arachidonic Acid, 99% pure from Porcine Liver (Sigma Aldrich) reconstituted in acetone 2 mg/20 μL (200 mg/mL).

Inhalation anesthesia: Isoflurane 3% (Baxter).

Blood Sample tubes: Microtainer tubes w/heparin (Becton Dickinson).

TNFα Elisa assay (R&D Science).

Experimental Procedure

Test compounds, positive control (arachidonic acid only) and standard (dexamethasone at 0.1 mg/kg) prepared in solutions of acetone, ethanol or aqueous ethanol, are applied to both sides of the right ear with an Eppendorf repipettor pipette, in a volume of 10 μL each side (20 μL total). 30 minutes later, 10 μL of arachidonic acid was applied to both sides of the right ear (20 μL total). One hour after the application of arachidonic acid, the mice are deeply anesthetized with isoflurane and a blood sample is taken via the orbital sinuses and placed in Microtainer tubes. The animals are then euthanized by CO₂ inhalation and the right ears removed at the base. A uniform plug of ear tissue is obtained using an 8 mm dermal punch. The earplugs are quickly weighed to the nearest 0.1 mg and then flash frozen for TNFα determination.

Statistical Analysis:

Group data is presented as means +/−SEM and p<0.05 is considered significant. Inter-group comparisons are carried out by unpaired student t tests (between two groups) or ANOVA (three or more groups) followed by post hoc Dunnet's test.

### Example 12

### Healthy Skin Assessment/Improvement of Skin Appearance

A double blind placebo-controlled clinical study is conducted on human female subjects ages 21-40 to assess the efficacy of compositions to affect tone and tactile properties of human skin. Subjects are directed to apply skin care compositions of the invention or carrier-matched placebo formulation to ½ of the face daily for 6 weeks. Scaling, moisturization, oiliness, smoothness, redness and blotchiness are recorded by instrumental measurements, by digital photography, and by self-assessment.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All patents and publications cited above are hereby incorporated by reference.

What is claimed is:

1. A compound represented by Formula I: 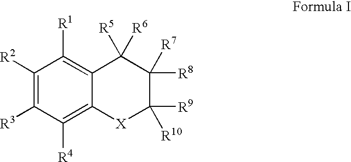
wherein,
X is O, S(O)₀₋₂, or NR;
R¹ and R⁴ are independently selected from the group consisting of hydrogen, alkyl, alkenyl, alkynyl, cycloalkyl, halogen, nitro, cyano, amino, aminosulfonyl, sulfanyl, aryl, heterocyclyl, hydroxy, alkoxy, carboxy, alkoxycarbonyl, and amido; with the proviso that no more than one of R¹ and R⁴ is hydrogen;
R² is selected from the group consisting of hydroxy, alkoxy, —O-alkenyl, —O-acyl, —O-alkylene-amino, —O—C(O)-alkylene-COOR^{b}, —O—C(O)-alkylene-amino, —O—C(O)-alkylene-heterocyclyl, —O-glucoside, —O-phosphoryl, —O-alkylene-phosphoryl, or —O—C(O)-AA, wherein AA is amino acid, or a di-, tri-, or tetra-peptide;
R³ is selected from the group consisting of alkyl, alkenyl, alkynyl, cycloalkyl, halogen, nitro, cyano, amino, aminosulfonyl, sulfanyl, aryl, heterocyclyl, alkoxy, carboxy, alkoxycarbonyl, and amido; or
R³ and R⁴ together with the atoms to which they are attached form a cycloalkyl ring, aryl ring or a heterocyclic ring;
R⁵ and R⁶ are independently selected from the group consisting of hydrogen, alkyl, cycloalkyl, hydroxy, —NR^{d}OR^{a}, and —NR^{d}—NR^{b}R^{c};
R⁷ and R⁸ are
independently selected from the group consisting of hydrogen, alkyl, cycloalkyl, —NR^{d}OR^{a}, and —NR^{d}—NR^{b}R^{c}; or
together with the carbon atom to which they are attached form a C═NOR^{a} or a C═N—NR^{b}R^{c} group;
R⁹ is selected from the group consisting of hydrogen, alkyl and cycloalkyl;
R¹⁰ is alkyl or cycloalkyl;
R is selected from the group consisting of hydrogen, alkyl, cycloalkyl, alkenyl, alkynyl, acyl, aminocarbonyl, heterocyclyl, and aryl;
R^{a} is selected from the group consisting of alkyl, cycloalkyl, alkenyl, acyl, heterocyclyl, and aryl; and
R^{b} and R^{c} are
independently selected from the group consisting of hydrogen, alkyl, cycloalkyl, alkenyl, acyl, aminocarbonyl, heterocyclyl and aryl; or
together with the nitrogen atom to which they are attached form an optionally substituted, saturated or unsaturated 3-8 membered ring optionally incorporating 1 to 3 N, O or S atoms; and
R^{d} is hydrogen or alkyl;
with the proviso that one of the following is present
R is OH, —NR^{d}OR^{a} or —NR^{d}—NR^{b}R^{c}; or
R is —NR^{d}OR^{a} or —NR^{d}—NR^{b}R^{c}; or
R⁷ and R⁸ together with the carbon atom to which they are attached form a C═NOR^{a} or a C═N—NR^{b}R^{c} group;
or single stereoisomers, mixtures of stereoisomers, or pharmaceutically acceptable salts thereof.

2. The compound of claim 1, wherein R² is hydroxy.

3. The compound of claim 2, wherein R¹, R³, and R⁴ are independently selected from the group consisting of hydrogen, halogen, and alkyl.

4. The compound of claim 1, wherein X is O.

5. The compound of claim 1, wherein X is S.

6. The compound of claim 1, wherein X is NR.

7. The compound of claim 2, wherein —CR⁷R⁸ is C═NOR^{a}.

8. The compound of claim 2, wherein —CR⁷R⁸ is C═N—NR^{b}R^{c}.

9. The compound of claim 2, wherein R⁵ is —NR^{d}OR^{a}.

10. The compound of claim 2, wherein R⁵ is —NR^{d}—NR^{b}R^{c}.

11. The compound of claim 2, wherein R⁵ is OH and R⁶ is hydrogen.

12. The compound of claim 2, wherein R⁷ is —NR^{d}OR^{a}.

13. The compound of claim 2, wherein R⁷ is —NR^{d}—NR^{b}R^{c}.

14. The compound of claim 2, wherein R¹, R³, and R⁴ are independently selected from the group consisting of hydrogen, halogen, and alkyl, and X is O.

15. The compound of claim 2, wherein R¹, R³, and R⁴ are independently selected from the group consisting of hydrogen, halogen, and alkyl, and X is S.

16. The compound of claim 2, wherein R¹, R³, and R⁴ are selected from the group consisting of hydrogen, halogen, or alkyl, and X is NR.

17. The compound of claim 16 wherein R is selected from the group consisting of aryl, heterocyclyl, and alkyl optionally substituted with amido, sulfonylamino or aminosulfonyl.

18. A pharmaceutical composition comprising a therapeutically effective amount of one or more compounds of claim 1 admixed with a pharmaceutically acceptable excipient.

19. A method of treating a subject with a lipoxygenase mediated condition comprising administering to said subject a therapeutically effective amount of a pharmaceutical composition of claim 18.

20. The method of claim 19, wherein the condition is selected from the group consisting of apoptosis in cancer cells including prostatic cancer, gastric cancer, breast cancer, pancreatic cancer, colorectal or esophageal cancer and airways carcinoma; diseases involving hypoxia or anoxia including atherosclerosis, myocardial infarction, cardiovascular disease, heart failure (including chronic and congestive heart failure), cerebral ischemia, retinal ischemia, myocardial ischemia, post surgical cognitive dysfunction and other ischemias; diseases involving inflammation, including diabetes, arterial inflammation, inflammatory bowel disease, Crohn's disease, renal disease, pre-menstrual syndrome, asthma, allergic rhinitis, gout, cardiopulmonary inflammation, rheumatoid arthritis, osteoarthritis, muscle fatigue; disorders of the airways including asthma, chronic bronchitis, human airway carcinomas, mucus hypersecretion, chronic obstructive pulmonary disease (COPD) pulmonary fibrosis caused by chemotherapy or other drugs, idiopathic pulmonary fibrosis, cystic fibrosis and adult respiratory distress syndrome; diseases involving central nervous system (CNS) disorders including psychiatric disorders including anxiety and depression; neurodegeneration and neuroinflammation including Alzheimer's, dementia and Parkinson's disease; peripheral neuropathy including spinal chord injury, head injury and surgical trauma, and allograft tissue and organ transplant rejection; diseases involving the autoimmune system including rheumatoid arthritis and diabetes; and disorders involving bone loss or bone formation.

21. The method of claim 19, wherein the condition is selected from the group consisting of dermatitis, including atopic, contact, and allergic dermatitis, xerosis, eczema, rosacea, seborrhea, psoriasis, atherosclerosis, thermal and radiation burns, acne, oily skin, wrinkles, excessive cellulite, excessive pore size, intrinsic skin aging, photo aging, photo damage, harmful UV damage, keratinization abnormalities, irritation including retinoid induced irritation, hirsutism, alopecia, dyspigmentation, inflammation due to wounds, scarring or stretch marks, loss of elasticity, and skin atrophy.

22. A method of treating a subject suffering from diabetes, arthritis, rheumatoid arthritis, chronic obstructive pulmonary disease (COPD), asthma, allergic rhinitis, or atherosclerosis comprising administering a therapeutically effective amount of a composition of claim 18.

23. A compound selected from 2,2,5,7,8-pentamethylchroman-4,6-diol; 2,2,7,8-tetramethylchroman-4,6-diol; 5,7-diethyl-2,2-dimethylchroman-4,6-diol; 5-ethyl-7-isopropyl-2,2-dimethylchroman-4,6-diol; and 7-isopropyl-2,2,5-trimethylchroman-4,6-diol; or stereoisomers, mixture of stereoisomers or pharmaceutically acceptable salts thereof.

24. A compound selected from 4-methoxyamino-2,2,5,7,8-pentamethyl-chroman-6-ol; 4-(methoxyamino)-2,2,7,8-tetramethylchroman-6-ol; 5,7-diethyl-4-(methoxyamino)-2,2,8-trimethylchroman-6-ol; 7-isopropyl-4-(methoxyamino)-2,2,5-trimethylchroman-6-ol; and 7-isopropyl-4-(methoxyamino)-2,2,5-trimethylchroman-6-ol; or stereoisomers, mixture of stereoisomers or pharmaceutically acceptable salts thereof.

25. A pharmaceutical composition comprising as the active component a compound represented by Formula IA: 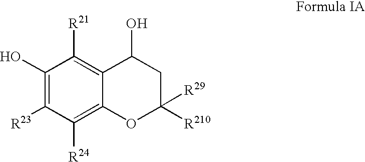
wherein,
R²¹, R²⁴ and R²⁹ are independently selected from the group consisting of hydrogen, alkyl and cycloalkyl; with the proviso that no more than one of R²¹ and R²⁴ is hydrogen and
R²³ and R²¹⁰ are independently alkyl or cycloalkyl;
or single stereoisomers, mixtures of stereoisomers, or pharmaceutically acceptable salts thereof; admixed with a pharmaceutically acceptable excipient.

26. The pharmaceutical composition of claim 25, wherein R²¹ and R²³ are C₁₋₄ alkyl, R²⁴ is hydrogen, and R²⁹ and R²¹⁰ are both methyl.

27. A method of treating a subject with a lipoxygenase mediated condition comprising administering to said subject a therapeutically effective amount of a composition of claim 25.

28. A method of treating a subject suffering from a condition selected from the group consisting of diabetes, arthritis, rheumatoid arthritis, chronic obstructive pulmonary disease (COPD), asthma, allergic rhinitis, or atherosclerosis comprising administering a therapeutically effective amount of a composition of claim 25.

29. A method of treating a subject suffering from a condition selected from the group consisting of dermatitis, eczema, or psoriasis comprising administering a therapeutically effective amount of a composition of claim 25.

30. A skin care composition comprising as the active component a compound represented by Formula IA: 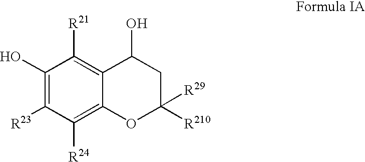
wherein,
R²¹, R²⁴ and R²⁹ are independently selected from the group consisting of hydrogen, alkyl and cycloalkyl; with the proviso that no more than one of R²¹ and R²⁴ is hydrogen and R²³ and R²¹⁰ are independently alkyl or cycloalkyl;
or single stereoisomers, mixtures of stereoisomers, or pharmaceutically acceptable salts thereof; admixed with a cosmetically acceptable carrier.

31. The skin care composition of claim 30, wherein R²³ and R²⁴ are independently C₁₋₄ alkyl, R²¹ is hydrogen or methyl, and R²⁹ and R²¹⁰ are both methyl.

32. The skin care composition of claim 30, further comprising at least one agent selected from the group consisting of:
(i) a skin protectant active ingredient selected from the group consisting of allantoin, aluminum hydroxide gel, calamine, cocoa butter, cod liver oil, colloidal oatmeal, dimethicone, glycerin, hard fat, kaolin, lanolin, mineral oil, petrolatum, soy products, sodium bicarbonate, topical starch, white petrolatum, zinc acetate, and/or zinc oxide;
(ii) an external analgesic, anesthetic or antipruritic ingredient selected from the group consisting of benzocaine, butamaben picrate, dibucaine, dibucaine hydrochloride, dimethiosoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium resorcinol, tripelennamine hydrochloride, aspirin, hydrocortisone, hydrocortisone acetate, and/or diphenydramine hydrochloride;
(iii) a keratolytic agent selected from the group consisting of salicylic acid or esters thereof, benzoyl peroxide, resorcinol, colloidal sulfur, selenium disulphide, sulfur and combinations thereof; and
(iv) a retinoid selected from the group consisting of retinol, retinoic acid and esters thereof.

33. The skin care composition of claim 32, wherein the agent is a retinoid.

34. The skin care composition of claim 32, wherein the agent is a soy product.

35. A method of treating a lipoxygenase mediated condition comprising administering a cosmetically effective amount of a skin care composition of claim 30, wherein the condition is selected from the group consisting of dermatitis, including atopic, contact, and allergic dermatitis, xerosis, eczema, rosacea, seborrhea, psoriasis, atherosclerosis, thermal and radiation burns, acne, oily skin, wrinkles, excessive cellulite, excessive pore size, intrinsic skin aging, photo aging, photo damage, harmful UV damage, keratinization abnormalities, irritation including retinoid induced irritation, hirsutism, alopecia, dyspigmentation, inflammation due to wounds, scarring or stretch marks, loss of elasticity, and skin atrophy.

36. The method of claim 35, wherein the condition is retinoid induced irritation.

37. The method of claim 35, wherein the condition is acne.

38. A skin care composition comprising as the active component 2,2,5,7,8-pentamethylchroman-4,6-diol, 2,2,7,8-tetramethylchroman-4,6-diol, and mixtures thereof admixed with a cosmetically acceptable carrier.

39. The skin care composition of claim 38, further comprising at least one agent selected from the group consisting of:
(i) a skin protectant active ingredient selected from the group consisting of allantoin, aluminum hydroxide gel, calamine, cocoa butter, cod liver oil, colloidal oatmeal, dimethicone, glycerin, hard fat, kaolin, lanolin, mineral oil, petrolatum, soy products, sodium bicarbonate, topical starch, white petrolatum, zinc acetate, and/or zinc oxide;
(ii) an external analgesic, anesthetic or antipruritic ingredient selected from the group consisting of benzocaine, butamaben picrate, dibucaine, dibucaine hydrochloride, dimethiosoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium resorcinol, tripelennamine hydrochloride, aspirin, hydrocortisone, hydrocortisone acetate, and/or diphenydramine hydrochloride;
(iii) a keratolytic agent selected from the group consisting of salicylic acid or esters thereof, benzoyl peroxide, resorcinol, colloidal sulfur, selenium disulphide, sulfur and combinations thereof; and
(iv) a retinoid selected from the group consisting of retinol, retinoic acid and esters thereof.

40. The skin care composition of claim 39, wherein the agent is a retinoid.

41. The skin care composition of claim 39, wherein the agent is a soy product.

42. A method of treating a lipoxygenase mediated condition comprising administering a cosmetically effective amount of a skin care composition of claim 38, wherein the condition is selected from the group consisting of dermatitis, including atopic, contact, and allergic dermatitis, xerosis, eczema, rosacea, seborrhea, psoriasis, atherosclerosis, thermal and radiation burns, acne, oily skin, wrinkles, excessive cellulite, excessive pore size, intrinsic skin aging, photo aging, photo damage, harmful UV damage, keratinization abnormalities, irritation including retinoid induced irritation, hirsutism, alopecia, dyspigmentation, inflammation due to wounds, scarring or stretch marks, loss of elasticity, and skin atrophy.

43. The method of claim 42, wherein the condition is retinoid induced irritation.

44. The method of claim 42, wherein the condition is acne.

45. A pharmaceutical composition comprising as the active component a compound represented by Formula IB: 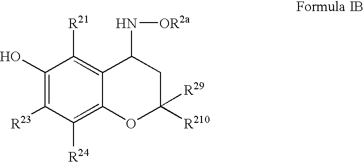
wherein,
R², R²⁴ and R²⁹ are independently selected from the group consisting of hydrogen, alkyl and cycloalkyl; with the proviso that no more than one of R²¹ and R²⁴ is hydrogen R²³ and R²¹⁰ are independently alkyl or cycloalkyl; and
R^{2a} is alkyl, cycloalkyl;
or single stereoisomers, mixtures of stereoisomers, or pharmaceutically acceptable salts thereof; admixed with a pharmaceutically acceptable excipient.

46. The pharmaceutical composition of claim 45, wherein R²¹ and R²³ are independently C₂₋₄ alkyl, R²⁴ is hydrogen, and R²⁹ and R²¹⁰ are both methyl.

47. A method of treating a subject with a lipoxygenase mediated condition comprising administering to said subject a therapeutically effective amount of a composition of claim 45.

48. A method of treating a subject suffering from diabetes, arthritis, rheumatoid arthritis, chronic obstructive pulmonary disease (COPD), asthma, allergic rhinitis, dermatitis, eczema, psoriasis or atherosclerosis comprising administering a therapeutically effective amount of a composition of claim 45.
